(19) United States
(12) Patent Application Publication
(10) Pub. No.: US 2024/0197438 A1
(43) Pub. Date: Jun. 20, 2024

(54) SYSTEM AND METHOD FOR LOCATING AND MANAGING PATIENT SUPPORT APPARATUS IN A HEALTHCARE FACILITY

(71) Applicant: UMANO MEDICAL INC., L'Islet (CA)

(72) Inventors: Anouer KEBIR, Saint-Jean-Chrysostome (CA); Jean-François GROU, Montréal (CA); Esther BERTHELOT, Lévis (CA); Jérôme MARCOTTE, Lévis (CA)

(73) Assignee: UMANO MEDICAL INC., L'Islet, QC (CA)

(21) Appl. No.: 18/552,997

(22) PCT Filed: Mar. 31, 2022
(86) PCT No.: PCT/IB2022/053030

§ 371 Date: Sep. 28, 2023

Related U.S. Application Data
(60) Provisional application No. 63/168,805, filed on Mar. 31, 2021.

(51) Int. Cl. A61B 90/98; A61G 7/05; H04W 64/00; H04W 84/12

(52) U.S. Cl. CPC A61B 90/98; A61G 7/05; H04W 64/006; A61G 2203/20; A61G 2205/60; H04W 84/12

(57) ABSTRACT

A method performed by a first device for locating a medical device within a medical facility includes determining, by the first device, that a second device is located in a same room as the first device, based on a first signal communicated between the first device and the second device; determining, by the first device, a distance to the at least one second device, based on a second signal communicated between the first device and the second device; and establishing communication with the second device. A device, a wireless stationary node, a location determining system and method, and a patient support apparatus are also described.

### FIELD

The present technology generally relates to a patient support apparatus in general and more specifically to systems and methods for managing a plurality of patient support apparatus in a healthcare facility. The technology also provides location detection systems useable in such managing systems.

### BACKGROUND

Location detection systems are known in the art for tracking the location of personnel and equipment in facilities. These systems have been specifically adapted for use in facilities such as healthcare facilities for tracking healthcare professionals, e.g., nurses and physicians, and for tracking equipment, e.g., hospital beds, patient monitoring devices, and the like. Some location detection systems utilize tags that periodically transmit a unique identification signal. Stationary receivers are located throughout the facility at known locations for receiving these identification signals. The stationary receivers are connected to a central computer, typically by wires, that processes the unique identification signals to determine a location of the asset associated with the tag.

Other location detection systems use transceivers positioned on board the patient support apparatuses and determine the locations of the patient support apparatuses based on signal strength data.

Managing systems for managing a plurality of patient support apparatuses in a healthcare facility have been proposed in the prior art, typically associated with location detection systems. Such managing systems are generally devised to provide a remote and centralized interface to ease monitoring and managing of the patient apparatuses present in the healthcare facility.

Today, one difficulty is the cost and complexity of the deployment of such managing systems. Specific receivers adequately distributed in the facility have to be installed and specifically adapted to the existing systems of the facility such as the nurse call system as a non-limitative example.

Moreover, depending of the configuration of a particular room accommodating several patients, additional locating devices may be required to provide sufficient spatial precision and differentiation between the medical equipment associated with each patient of the particular room. In some cases, costs can become prohibitive and/or performances of the managing systems may be impacted negatively, which may lead to a managing system that is not effective nor accurate enough. Furthermore, during this cumbersome installation, the room cannot be used for patient's care.

Besides, once installed, such managing systems may be complex to operate. Some relies on specific manual operations that have to be performed by a caregiver directly at the bed, which is time-consuming and can lead to errors or difficulties to configure the bed correctly in the managing system.

It should also be appreciated that various medical devices of various manufacturers as well as various dedicated computer systems often coexist in an existing healthcare facility, leading to added complexity and costs to the managing system, or even the impossibility to manage all data associated with a specific patient in an easy integrated manner.

There is therefore still a need for a managing system that would be easy to implement in a cost-effective manner.

There is also still a need for an improved locating system that would be precise enough to offer improved locating performances and reliability, while being easy to implement in a cost-effective manner.

### SUMMARY

It is an object of the present technology to ameliorate at least some of the inconveniences present in the prior art.

It is an object of the present technology to determine the location of a portable identification device in a medical facility by determining the nearest stationary node in the same room as the portable identification device.

In accordance with a first broad aspect, a method performed by a first device includes: determining, by a first device, that a second device is located in a same room as the first device, based on a first signal communicated between the first device and the second device: determining, by the first device, a distance to the at least one second device, based on a second signal communicated between the first device and the second device: and establishing communication with the second device.

Optionally, in any of the previous aspects, determining, by the first device, a distance to the second device includes determining a respective distance to each one of a plurality of second devices.

Optionally, in any of the previous aspects, establishing communication with the second device includes establishing communication with a nearest one of the plurality of second devices that is located in the same room as the first device.

Optionally, in any of the previous aspects, the first signal includes a device identifier (ID) corresponding to the second device.

Optionally, in any of the previous aspects, the device ID is a common device ID to all second devices in the same room.

Optionally, in any of the previous aspects, the first signal comprises an infrared signal.

Optionally, in any of the previous aspects, the first signal comprises an ultrasonic signal.

Optionally, in any of the previous aspects, the second signal comprises an ultra-wideband (UWB) signal.

Optionally, in any of the previous aspects, the second signal comprises an ultrasonic signal.

Optionally, in any of the previous aspects, the first device is, or is attached to, a piece of medical equipment.

Optionally, in any of the previous aspects, the first device is, or is attached to, a hospital bed.

Optionally, in any of the previous aspects, the second device is at a fixed location in the room, and the room is located in a hospital.

In accordance with a second broad aspect, a first device includes at least one processor, and at least one transceiver connected to the processor. The processor is configured to: determine that a second device is located in a same room as the first device, based on a first signal communicated between the at least one transceiver and the second device; determine a distance to the at least one second device, based on a second signal communicated between at least one transceiver and the second device; and establish communication with the second device.

Optionally, in any of the previous aspects, determining, by the first device, a distance to the second device includes determining a respective distance to each one of a plurality of second devices.

Optionally, in any of the previous aspects, establishing communication with the second device includes establishing communication with a nearest one of the plurality of second devices that is located in the same room as the first device.

Optionally, in any of the previous aspects, the first signal includes a device identifier (ID) corresponding to the second device.

Optionally, in any of the previous aspects, the device ID is a common device ID to all second devices in the same room.

Optionally, in any of the previous aspects, the first signal comprises an infrared signal.

Optionally, in any of the previous aspects, the first signal comprises an ultrasonic signal.

Optionally, in any of the previous aspects, the second signal comprises an ultra-wideband (UWB) signal.

Optionally, in any of the previous aspects, the second signal comprises an ultrasonic signal.

Optionally, in any of the previous aspects, the first device is, or is attached to, a piece of medical equipment.

Optionally, in any of the previous aspects, the first device is, or is attached to, a hospital bed.

Optionally, in any of the previous aspects, the second device is at a fixed location in the room, and the room is located in a hospital.

In accordance with a third broad aspect, a wireless stationary node for determining a location of a portable identification device in a facility, includes a processor; and at least one transceiver operatively connected to the processor. The at least one transceiver is operable to communicate at least one signal with the portable identification device. The processor is configured to determine a distance of the portable identification device from the wireless stationary node based on the at least one signal and to determine an angle of arrival of the at least one signal. The processor is configured to determine a location of the portable identification device based on the determined distance and the determined angle of arrival.

Optionally, in any of the previous aspects, the at least one signal comprises an ultrasonic signal or an ultra-wideband (UWB) signal.

Optionally, in any of the previous aspects, the at least one signal comprises a first signal and a second signal. The processor is configured to determine the distance of the portable identification device from the stationary node based on the first signal and to determine an angle of arrival of the second signal.

Optionally, in any of the previous aspects, the at least one transceiver comprises an ultra-wideband (UWB) transceiver for communicating the first signal.

Optionally, in any of the previous aspects, the at least one transceiver comprises an ultra-wideband (UWB) transceiver for communicating the first signal and the second signal.

Optionally, in any of the previous aspects, the at least one transceiver comprises an ultrasonic transceiver for communicating the first signal.

Optionally, in any of the previous aspects, the at least one transceiver comprises an infrared transceiver for communicating the second signal.

Optionally, in any of the previous aspects, the at least one transceiver comprises an ultrasonic transceiver for communicating the second signal.

Optionally, in any of the previous aspects, the at least one transceiver comprises a Bluetooth transceiver for communicating the second signal.

Optionally, in any of the previous aspects, the processor is configured to determine the distance of the portable identification device based on a received signal strength of the first signal.

Optionally, in any of the previous aspects, the distance of the portable identification device is determined based on a time of flight of the second signal.

Optionally, in any of the previous aspects, the angle of arrival of the second signal is determined based on a phase difference of arrival of the second signal at two or more receive antennas of the at least one transceiver.

Optionally, in any of the previous aspects, the wireless stationary node is further configured for detecting only a portable identification device that is located in a same room in which the wireless stationary node is installed.

Optionally, in any of the previous aspects, the wireless stationary node further includes one of an infrared module and an ultrasound module for detecting only the portable identification device that is located in the same room in which the wireless stationary node is installed.

Optionally, in any of the previous aspects, the portable identification device is, or is attached to, a piece of medical equipment.

Optionally, in any of the previous aspects, the portable identification device is, or is attached to, a hospital bed.

Optionally, in any of the previous aspects, the wireless stationary node is at a fixed location in a room of a hospital.

Optionally, in any of the previous aspects, the first signal is communicated with a first portable transceiver of the portable identification device: and the second signal is communicated with a second portable transceiver of the portable identification device.

In accordance with a fourth broad aspect, a location determination system, for determining location of a plurality of portable identification devices in a healthcare facility having a plurality of rooms, includes a plurality of wireless stationary nodes of any of the previous aspects. Each of the wireless stationary nodes has a unique identifier. The plurality of wireless stationary nodes are distributed in the rooms of the healthcare facility. The location of each of the portable identification devices is determined using a nearest wireless stationary node to the portable identification device in the facility. A remote server is operatively connected to the wireless stationary nodes and operable to receive the respective location of each of said portable identification devices. The remote server embeds a real-time locating system (RTLS) application configured for visual representation of the location of each of the portable identification devices in the facility.

Optionally, in any of the previous aspects, the facility comprises a plurality of patient support apparatus positions, each of said patient support apparatus positions being equipped with one of the plurality of said wireless stationary nodes.

Optionally, in any of the previous aspects, each of the wireless stationary nodes further includes an orientation detection module adapted to detect an orientation of the corresponding portable identification device.

Optionally, in any of the previous aspects, each of the wireless stationary nodes further includes a geofencing module adapted for detecting only the portable identification devices that are located in a same room in which the wireless stationary node is installed.

Optionally, in any of the previous aspects, the remote server embeds a managing application configured for pairing together each of the corresponding portable identification devices associated with a patient based on the location thereof.

In accordance with a fifth broad aspect, a patient support apparatus includes a support surface for supporting a patient: a processor; and at least one transceiver operatively connected to the processor, the at least one transceiver being operable to communicate at least one signal with a wireless stationary node. The processor is configured to determine a distance of the patient support apparatus from the wireless stationary node based on the at least one signal and to determine an angle of arrival of the at least one signal. The processor is configured to determine a location of the portable identification device based on the determined distance and the determined angle of arrival.

Optionally, in any of the previous aspects, the at least one signal comprises an ultrasonic signal or an ultra-wideband (UWB) signal.

Optionally, in any of the previous aspects, the at least one signal includes a first signal and a second signal. The processor is configured to determine the distance of the patient support apparatus from the stationary node based on the first signal and to determine an angle of arrival of the second signal.

Optionally, in any of the previous aspects, the at least one transceiver comprises an ultra-wideband (UWB) transceiver for communicating the first signal.

Optionally, in any of the previous aspects, the at least one transceiver comprises an ultra-wideband (UWB) transceiver for communicating the first signal and the second signal.

Optionally, in any of the previous aspects, the at least one transceiver comprises an ultrasonic transceiver for communicating the first signal.

Optionally, in any of the previous aspects, the at least one transceiver comprises an infrared transceiver for communicating the second signal.

Optionally, in any of the previous aspects, the at least one transceiver comprises an ultrasonic transceiver for communicating the second signal.

Optionally, in any of the previous aspects, the at least one transceiver comprises a Bluetooth transceiver for communicating the second signal.

Optionally, in any of the previous aspects, the processor is configured to determine the distance of the patient support apparatus from the wireless stationary node based on a received signal strength of the first signal.

Optionally, in any of the previous aspects, the distance of the patient support apparatus from the wireless stationary node is determined based on a time of flight of the second signal.

Optionally, in any of the previous aspects, the angle of arrival of the second signal is determined based on a phase difference of arrival of the second signal at two or more receive antennas of the at least one transceiver.

Optionally, in any of the previous aspects, the patient support apparatus is further configured for detecting only a wireless stationary node that is located in a same room in which the patient support apparatus is located.

Optionally, in any of the previous aspects, The patient support apparatus further includes one of an infrared module and an ultrasound module for detecting only the wireless stationary node that is located in a same room in which the patient support apparatus is located.

Optionally, in any of the previous aspects, the patient support apparatus is, or is attached to, a hospital bed.

Optionally, in any of the previous aspects, the wireless stationary node is at a fixed location in a room of a hospital.

Optionally, in any of the previous aspects, the first signal is communicated with a first portable transceiver of the wireless stationary node: and the second signal is communicated with a second portable transceiver of the wireless stationary node.

In accordance with a sixth broad aspect, a location determination method for determining a location of a plurality of portable identification devices in a facility, each of said portable identification devices comprising a first portable transceiver and a second portable transceiver, includes: exchanging first signals between a corresponding wireless stationary node and a corresponding one of said portable identification devices in the vicinity of said wireless stationary node: exchanging second signals between said corresponding wireless stationary node and said corresponding one of said portable identification devices in the vicinity of said wireless stationary node: and upon reception of said first signals and said second signals by the corresponding one of said wireless stationary nodes: determining, by a processor of the corresponding wireless stationary node, a distance of the corresponding portable identification device from the corresponding wireless stationary node based on the exchanged first signals: determining, by the processor of the corresponding wireless stationary node, an angle of arrival of the exchanged second signals from the corresponding portable identification device to the corresponding wireless stationary node: determining a precise location of the corresponding portable identification device based on the determined precise distance and the determined precise angle of arrival; and providing the determined precise location to a real-time locating system (RTLS) application for visual representation of the location of each of the portable identification devices in the facility.

Optionally, in any of the previous aspects, the location of each of said portable identification devices is determined using a nearest wireless stationary node to the corresponding portable identification device in the facility.

Optionally, in any of the previous aspects, the first signal comprises an ultra-wideband (UWB) signal and the second signal comprises a Bluetooth signal.

Additional and/or alternative features, aspects and advantages of implementations of the present technology will become apparent from the following description the accompanying drawings and the appended claims.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1 depicts a schematic diagram of a wireless stationary node in conjunction with a location detection system, in accordance with one embodiment;

FIG. 2A is a perspective view of a wireless stationary node, according to one embodiment;

FIG. 2B is a perspective view of another wireless stationary node, according to another embodiment;

FIG. 3 is a schematic perspective diagram of a healthcare facility provided with wireless stationary nodes: in accordance with one embodiment;

FIG. 4 shows an exemplary location detection system using wireless stationary nodes powered from the hospital power grid, according to one embodiment;

FIG. 5 shows another exemplary location detection system using wireless stationary nodes powered from Power over Ethernet (POE), according to another embodiment;

FIG. 6 is a schematic diagram of a portable identification device, according to one embodiment;

FIG. 7 is a schematic diagram of an identification device for use on a patient support apparatus, according to one embodiment;

FIG. 8 is a schematic diagram of a patient support apparatus provided with an identification device, according to one embodiment;

FIG. 9 is a diagram of a system for wirelessly connecting a patient support apparatus to an existing nurse call system, according to one embodiment;

FIG. 10 schematically illustrates a software application for a system using wireless stationary nodes, according to one embodiment;

FIG. 11 shows a location determination system, according to one embodiment;

FIG. 12 shows another location determination system, according to another embodiment;

FIG. 13 schematically illustrates another software application for a system using wireless stationary nodes, according to another embodiment;

FIG. 14 is a diagram of a system for estimating positions of identification devices, according to one embodiment;

FIG. 15 schematically illustrates another software application for a system using wireless stationary nodes, according to another embodiment;

FIG. 16 is a diagram of a system for providing precise positions of identification devices, according to one embodiment;

FIG. 17 schematically illustrates another software application for a system using wireless stationary nodes, according to another embodiment;

FIG. 18 shows a managing system configured to exchange wirelessly and automatically bidirectional data between medical equipment and various computer systems, according to another embodiment;

FIG. 19 shows another managing system configured to exchange wirelessly and automatically bidirectional data between medical equipment and various computer systems, according to another embodiment; and

FIG. 20 shows a workflow application, according to one embodiment.

### DETAILED DESCRIPTION

The examples and conditional language recited herein are principally intended to aid the reader in understanding the principles of the present technology and not to limit its scope to such specifically recited examples and conditions. It will be appreciated that those skilled in the art may devise various arrangements which, although not explicitly described or shown herein, nonetheless embody the principles of the present technology and are included within its spirit and scope.

### Definitions

In the context of the present specification, a “server” is a computer program that is running on appropriate hardware and is capable of receiving requests (e.g., from electronic devices) over a network (e.g., a communication network), and carrying out those requests, or causing those requests to be carried out. The hardware may be one physical computer or one physical computer system, but neither is required to be the case with respect to the present technology. In the present context, the use of the expression a “server” is not intended to mean that every task (e.g., received instructions or requests) or any particular task will have been received, carried out, or caused to be carried out, by the same server (i.e., the same software and/or hardware): it is intended to mean that any number of software elements or hardware devices may be involved in receiving/sending, carrying out or causing to be carried out any task or request, or the consequences of any task or request: and all of this software and hardware may be one server or multiple servers, both of which are included within the expressions “at least one server” and “a server”.

In the context of the present specification, “electronic device” is any computing apparatus or computer hardware that is capable of running software appropriate to the relevant task at hand. Thus, some (non-limiting) examples of electronic devices include general purpose personal computers (desktops, laptops, netbooks, etc.), mobile computing devices, smartphones, and tablets, and network equipment such as routers, switches, and gateways. It should be noted that an electronic device in the present context is not precluded from acting as a server to other electronic devices. The use of the expression “an electronic device” does not preclude multiple electronic devices being used in receiving/sending, carrying out or causing to be carried out any task or request, or the consequences of any task or request, or steps of any method described herein. In the context of the present specification, a “client device” refers to any of a range of end-user client electronic devices, associated with a user, such as personal computers, tablets, smartphones, and the like.

In the context of the present specification, the expression “computer readable storage medium” (also referred to as “storage medium” and “storage”) is intended to include non-transitory media of any nature and kind whatsoever, including without limitation RAM, ROM, disks (CD-ROMs, DVDs, floppy disks, hard drivers, etc.), USB keys, solid state-drives, tape drives, etc. A plurality of components may be combined to form the computer information storage media, including two or more media components of a same type and/or two or more media components of different types.

In the context of the present specification, a “database” is any structured collection of data, irrespective of its particular structure, the database management software, or the computer hardware on which the data is stored, implemented or otherwise rendered available for use. A database may reside on the same hardware as the process that stores or makes use of the information stored in the database or it may reside on separate hardware, such as a dedicated server or plurality of servers.

In the context of the present specification, the expression “information” includes information of any nature or kind whatsoever capable of being stored in a database. Thus, information includes, but is not limited to audiovisual works (images, movies, sound records, presentations etc.), data (location data, numerical data, etc.), text (opinions, comments, questions, messages, etc.), documents, spreadsheets, lists of words, etc.

In the context of the present specification, unless expressly provided otherwise, an “indication” of an information element may be the information element itself or a pointer, reference, link, or other indirect mechanism enabling the recipient of the indication to locate a network, memory, database, or other computer-readable medium location from which the information element may be retrieved. For example, an indication of a document could include the document itself (i.e. its contents), or it could be a unique document descriptor identifying a file with respect to a particular file system, or some other means of directing the recipient of the indication to a network location, memory address, database table, or other location where the file may be accessed. As one skilled in the art would recognize, the degree of precision required in such an indication depends on the extent of any prior understanding about the interpretation to be given to information being exchanged as between the sender and the recipient of the indication. For example, if it is understood prior to a communication between a sender and a recipient that an indication of an information element will take the form of a database key for an entry in a particular table of a predetermined database containing the information element, then the sending of the database key is all that is required to effectively convey the information element to the recipient, even though the information element itself was not transmitted as between the sender and the recipient of the indication.

In the context of the present specification, the expression “communication network” is intended to include a telecommunications network such as a computer network, the Internet, a telephone network, a Telex network, a TCP/IP data network (e.g., a WAN network, a LAN network, etc.), and the like. The term “communication network” includes a wired network or direct-wired connection, and wireless media such as acoustic, radio frequency (RF), infrared and other wireless media, as well as combinations of any of the above.

In the context of the present specification, the words “first”, “second”, “third”, etc. have been used as adjectives only for the purpose of allowing for distinction between the nouns that they modify from one another, and not for the purpose of describing any particular relationship between those nouns. Thus, for example, it should be understood that, the use of the terms “first server” and “third server” is not intended to imply any particular order, type, chronology, hierarchy or ranking (for example) of/between the server, nor is their use (by itself) intended to imply that any “second server” must necessarily exist in any given situation. Further, as discussed herein in other contexts, reference to a “first” element and a “second” element does not preclude the two elements from being the same actual real-world element. Thus, for example, in some instances, a “first” server and a “second” server may be the same software and/or hardware, in other cases they may be different software and/or hardware.

Furthermore, as an aid to understanding, the following description may describe relatively simplified implementations of the present technology. As persons skilled in the art would understand, various implementations of the present technology may be of a greater complexity.

In some cases, what are believed to be helpful examples of modifications to the present technology may also be set forth. This is done merely as an aid to understanding, and, again, not to define the scope or set forth the bounds of the present technology. These modifications are not an exhaustive list, and a person skilled in the art may make other modifications while nonetheless remaining within the scope of the present technology. Further, where no examples of modifications have been set forth, it should not be interpreted that no modifications are possible and/or that what is described is the sole manner of implementing that element of the present technology.

Moreover, all statements herein reciting principles, aspects, and implementations of the present technology, as well as specific examples thereof, are intended to encompass both structural and functional equivalents thereof, whether they are currently known or developed in the future. Thus, for example, it will be appreciated by those skilled in the art that any block diagrams herein represent conceptual views of illustrative circuitry embodying the principles of the present technology. Similarly, it will be appreciated that any flowcharts, flow diagrams, state transition diagrams, pseudo-code, and the like represent various processes which may be substantially represented in computer-readable media and so executed by a computer or processor, whether or not such computer or processor is explicitly shown.

The functions of the various elements shown in the figures, including any functional block labeled as a “processor” or a “graphics processing unit”, may be provided through the use of dedicated hardware as well as hardware capable of executing software in association with appropriate software. When provided by a processor, the functions may be provided by a single dedicated processor, by a single shared processor, or by a plurality of individual processors, some of which may be shared. In some non-limiting embodiments of the present technology, the processor may be a general purpose processor, such as a central processing unit (CPU) or a processor dedicated to a specific purpose, such as a graphics processing unit (GPU). Moreover, explicit use of the term “processor” or “controller” should not be construed to refer exclusively to hardware capable of executing software, and may implicitly include, without limitation, digital signal processor (DSP) hardware, network processor, application specific integrated circuit (ASIC), field programmable gate array (FPGA), read-only memory (ROM) for storing software, random access memory (RAM), and non-volatile storage. Other hardware, conventional and/or custom, may also be included.

Software modules, or simply modules which are implied to be software, may be represented herein as any combination of flowchart elements or other elements indicating performance of process steps and/or textual description. Such modules may be executed by hardware that is expressly or implicitly shown.

With these fundamentals in place, we will now consider some non-limiting examples to illustrate various implementations of aspects of the present technology.

### Wireless Stationary Node

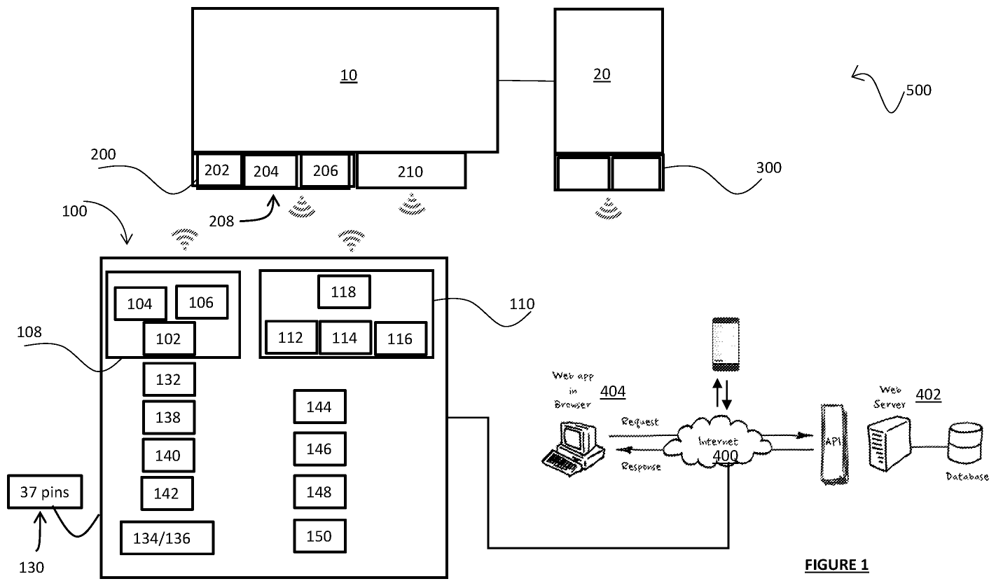

Referring to FIG. 1, there is shown a wireless stationary node 100 for determining a location of a portable identification device in a facility, in accordance with one embodiment of the present technology. The wireless stationary node 100 is also referred to as a wireless anchor point, as it will become apparent below. It will be appreciated that, in a healthcare application in a healthcare facility, a plurality of wireless stationary nodes 100 may be used. The location of the wireless stationary nodes 100 in the facility is not limited, and depending on the components of the wireless stationary node 100, the wireless stationary node 100 may be located on a wall, ceiling, floor or at least partially within a wall, ceiling, or floor of the facility. As a non-limiting example, one or more wireless nodes such as the wireless node 100 may be located in a room of a hospital.

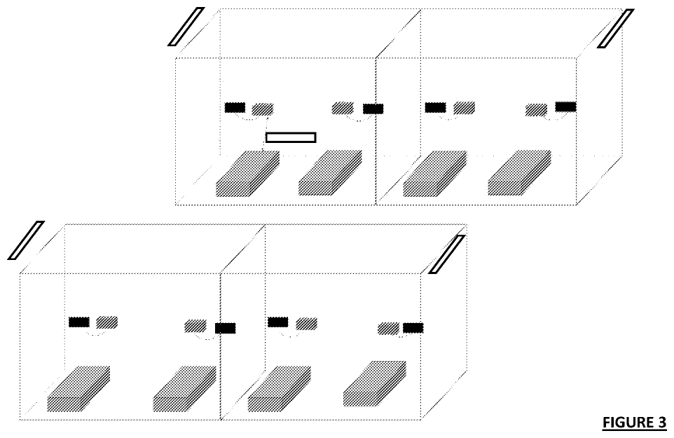

In one embodiment, the hospital has a plurality of rooms devised to receive one or a plurality of patients and thus provides one or several predetermined patient support apparatus positions. In one embodiment, each of the predetermined patient support apparatus positions is equipped with a single one of the plurality of the wireless stationary nodes 100, as illustrated in FIG. 3. In a further embodiment, the wireless stationary node 100 is mounted on the vertical wall located behind the head of the patient support apparatus, at a height enabling to minimize distance between the wireless stationary node and the electronics of the patient support apparatus, as it will become apparent below.

Still referring to FIG. 1, in the illustrated exemplary embodiment, a first portable identification device 200 is mounted on the frame of a patient support apparatus 10, as described in more detailed thereinafter, while a second portable identification device 300 is mounted on a cardiac monitor 20 connected to the patient lying in the patient support apparatus 10.

The wireless stationary node 100 is configured to exchange various signals with portable identification devices in the vicinity thereof, such as the portable identification devices 200 and 300 of FIG. 1.

The wireless stationary node 100 has a processor 102, a first transceiver 104 operatively connected to the processor 102 and a second transceiver 106 operatively connected to the processor 102. The first transceiver 104 is operable to exchange, i.e. transmit and/or receive, first signals with a first portable transceiver 204 of the portable identification device 200. The second transceiver 106 is operable to exchange, i.e. transmit and/or receive, second signals with a second portable transceiver 206 of the portable identification device 200. The processor 102, the first transceiver 104 and the second transceiver 106 will be referred as the location module 108 thereinafter. In the illustrated embodiment, the wireless signals are represented with a known Wi-Fi symbol but it should be mentioned that the wireless signals can be of several types, UWB, Bluetooth or Wi-Fi types for non-limitative examples, as it will become apparent below.

In one embodiment, the first transceiver 104 has a UWB transceiver exchanging UWB signals and the second transceiver 106 has a Bluetooth transceiver exchanging Bluetooth signals. In the following description of exemplary embodiments, the first transceiver 104 will generally be referred to as the UWB transceiver 104 but it should be understood that other types of transceivers could be used. Similarly, the second transceiver 106 will generally be referred to as the Bluetooth transceiver 106 but it should be understood that other types of transceivers could be used, as it should become apparent upon reading the present description. For example, in an alternative embodiment, each of the first transceiver and the second transceiver may have a distinct UWB transceiver. In this example, the determined distance may be determined according to signal strength, time of flight or other known techniques.

In one embodiment, the processor 102 is embedded in a microcontroller which is further provided with at least one non-transitory computer-readable storage medium (not shown). The microcontroller is operatively connected to the different components of the wireless stationary node 100, i.e. the UWB transceiver 104, the Bluetooth transceiver 106, and additional components that may be added for a particular application, as detailed below. The microcontroller is configured to communicate with and/or operate the other components of the wireless stationary node 100.

The processor 102 is adapted to determine a precise distance of the portable identification device 200 from the stationary node 100 based on the exchanged UWB signals and to further determine a precise angle of arrival of the exchanged Bluetooth signals. The processor 102 is further adapted to determine a precise location of the portable identification device 200 based on the determined precise distance and the determined precise angle of arrival, as it will become apparent to the skilled addressee upon reading the present description.

The angle of arrival of the received signal may be determined by any suitable method, such as by determining the phase difference of the received signal at two spaced-apart antennas of the receiving device. As it should be understood, the determined angle of arrival would allow to determine whether the portable identification device 200 is generally in front of the stationary node 100 and consequently in the same room, or behind the stationary node 100 and consequently in a distinct room.

In one embodiment, the processor 102, the UWB transceiver 104 and the Bluetooth transceiver 106 are integrated in a single chip. The single chip has a first and a second integrated antenna, the first antenna being adapted for UWB communication and the second antenna being adapted for Bluetooth communication. In one embodiment, a chip DWM1001C from Decawave, Ireland, may be used. Such chip enables the wireless stationary node 100 to work as a UWB anchor and/or a Bluetooth anchor.

It should be understood that either or both of the first transceiver 104 and the second transceiver 106 could instead be implemented as either a transmitter or a receiver, or as a combination transmitter-receiver. The term “transceiver” as used herein should be understood to encompass these implementations.

In one embodiment, the wireless stationary node 100 uses only a single transceiver 104 for exchanging signals with the portable identification device 200. In this embodiment, the first and second signals may be of the same type, corresponding to the type of transceiver 104. For example, if the transceiver 104 is a UWB transceiver, the first and second signals are first and second UWB signals. If the transceiver 104 is an ultrasonic transceiver, the first and second signals are first and second ultrasonic signals. In another embodiment, the single transceiver 104 transmits only one signal, for example an UWB signal or an ultrasonic signal, that can be used to determine both the distance to the portable identification device 200 and the angle of arrival of the signal. The angle of arrival of the received signal may be determined by any suitable method, such as by determining the phase difference of the received signal at two spaced-apart antennas of the receiving device.

In one embodiment, the wireless stationary node 100 is further adapted for wirelessly connecting a patient support apparatus 10 to a facility network. Accordingly, the wireless stationary node 100 is further provided with a communication module 110 adapted to exchange data, i.e. transmit and receive, with a facility network 400. In the illustrated embodiment, the communication module 110 has a processor 112, a Wi-Fi transceiver 114, a Bluetooth transceiver 116 and an Ethernet interface 118 but it should be understood that one or two of the Wi-Fi transceiver 114, the Bluetooth transceiver 116 and the Ethernet interface 118 may be omitted. In a further embodiment, the communication module 110 also has a UWB transceiver. In still a further embodiment and as it should become apparent below, the UWB transceiver may be used for data communication. In one embodiment, a chip ESP32 from Espressif Systems may be used for that purpose. This chip enables Wi-Fi/Ethernet/BLE (Bluetooth Low Energy) communication with a single integrated antenna for WI-Fi/BLE communication.

In this embodiment, the communication module 110 of the wireless stationary node thus enables to receive and send data from and to a patient support apparatus 10 connected thereto to enable a bidirectional communication of data between the facility network 400 and associated healthcare computer systems and the patient support apparatus 10. Data may be exchanged through Wi-Fi signals or alternatively through UWB signals, according to a specific application. Data exchange through UWB signals may be preferred in the case where interferences have to be minimized. In a further embodiment, it may be considered to exchange data through an Ethernet connection between the wireless stationary node 100 and the patient support apparatus 10.

In this embodiment, the patient support apparatus 10 is equipped with a portable identification device 200. The UWB portable transceiver 204 and the Bluetooth portable transceiver 206 of the portable identification device 200 are embedded in a location module 208 that is affixed to the frame of the patient support apparatus 10. Moreover, the patient support apparatus 10 is further provided with a communication module 210 that is adapted for automatic connection to the communication module 110 of the wireless stationary node 100.

In some embodiments, the wireless stationary node 100 is further provided with a nurse call interface 130 comprising a connector, typically a 37 pins connector but other types of connector could be considered, for connecting to an existing nurse call system of the healthcare facility. In further embodiments, the wireless stationary node 100 further has nurse call relays 132 configured to transfer nurse calls to the nurse call system of the healthcare facility.

In one embodiment, the wireless stationary node 100 is used to connect the patient support apparatus 10 to the hospital network 400 according to the method described in PCT application WO2021074895 of the same applicant, which is hereby incorporated by reference. The skilled addressee will also appreciate that alternate methods could also be used.

As previously mentioned, the above-described wireless stationary node 100 enables to provide location of various medical equipment with a great precision. However, in some circumstances due to a specific facility configuration using a great number of wireless stationary nodes 100 in a reduced space, inventors appreciated that the additional combined use of geofencing technology may be advantageous.

Accordingly, in one embodiment, the wireless stationary node 100 is further provided with one of an infrared module 134 and/or an ultrasound module 136 for detecting only a portable identification device that is located in a same room in which the wireless stationary node 100 is installed. This may ensure that the portable identification device that is detected by the wireless stationary node 100 is actually in the same room and not in an adjacent room on the same separation wall. In one embodiment, the infrared module 134 has only a receiver or only a transmitter or both of them. The portable identification device 200 is also provided with a corresponding infrared module 234 adapted to communicate with the infrared module 134 of the wireless stationary node 100, i.e. it has only a receiver, only a transmitter or both. Alternatively, it could also be envisaged to use an infrared module 134 having both a transmitter and a receiver adapted to detect infrared signals reflected by object in the close vicinity thereof. For example, the infrared transceiver GP2Y0A41SK0F of Sharp Company may be used.

In one embodiment, the infrared module 134 of the stationary node 100 is provided with multiple infrared transceivers arranged in different directions, for example 5 infrared transceivers arranged in 5 directions, to provide an enlarged angle of view and ease transmission or detection of the infrared signals exchanged with the portable identification device. In such an embodiment, the infrared detection is simplified while still providing the required information. The patient support apparatus may be conveniently detected in any configuration thereof, for example in the lowermost position of the patient support surface, the uppermost position of the patient support surface, or even when the patient surface is angle with respect to the horizontal or the patient support apparatus is not in longitudinal alignment with the stationary node 100.

The skilled addressee will appreciate that the infrared technology could be replaced by ultrasound technology. For example, the ultrasonic distance sensor US100 from Adafruit Company may be used. In one embodiment, the ultrasonic module may be provided with multiple ultrasonic transceivers arranged in multiple directions, as previously described with respect to the infrared module 134.

Alternate solutions to know if the detected identification portable device is actually in the same room than the wireless stationary node 100 may also be implemented. One solution relies on the detection of the orientation of the portable identification device with respect to the wireless stationary node 100 with which UWB and/or Bluetooth location signals are exchanged.

Accordingly, in one embodiment, the wireless stationary node 100 has an orientation detection module 138 adapted to detect an orientation of the portable identification device. The orientation detection module 138 has at least one of a Bluetooth transmitter and a Bluetooth receiver implemented according to recent BLE protocol 5.2. Both Bluetooth transmitter and receiver may be implemented. The portable identification device 200 is also provided with corresponding Bluetooth transmitter and/or receiver 238, as detailed below.

In an alternate embodiment, the orientation detection module 138 uses the location detection UWB transceiver 104 in combination with an additional antenna associated therewith. The orientation detection module 138 is further provided with a UWB chip having a processor which is operable to determine the orientation of the portable identification device based on the UWB signals exchanged with the two UWB antennas. The UWB chip and his processor are operatively connected to the processor 102 of the wireless stationary node 100 for the determination of the precise position. In one embodiment, the previously mentioned chip DWM1001C from Decawave may be replaced with an arrangement of two chips DWM1000 from Decawave with two integrated customized antennas. Alternatively, the chip DWM 1002 from Decawave provided with two integrated antennas may be used.

In another alternate embodiment, the processor 102 of the wireless stationary node 100 is further adapted to monitor signal strength of Bluetooth signals of the Bluetooth transceiver 116 of the communication module 110. Thus, in the case where the wireless stationary node 100 is provided with the communication module 110 for connecting a patient support apparatus 10 to the hospital network 400, the orientation detection of the portable identification device may be implemented without requiring additional materials and without significant costs.

The wireless stationary node 100 may be provided with additional modules that may be used according to various combinations depending of a specific application.

Still referring to FIG. 1, in some embodiments, the wireless stationary node 100 has an AC/DC power source 140 for powering directly the wireless stationary node 100 from the power grid in the case where Wi-Fi or Ethernet are used for the communication of patient and/or bed related data. In some embodiments, the wireless stationary node 100 has a RJ45 port 142 enabling Ethernet communication with the hospital network 400 and/or providing Power over Ethernet (POE) to the wireless stationary node 100. In some embodiments, the wireless stationary node 100 has a Power over Ethernet power circuit 144 for providing power thereto from the RJ45 port 142. In some embodiments, the wireless stationary node 100 has a USB-hub circuit 146 enabling to program the location module 108 and the communication module 110 independently from each other. In some embodiments, the wireless stationary node 100 has a USB C port 148 enabling to configurate and update the wireless stationary node 100 from a PC or mobile application, as described in more details below. In some embodiments, the wireless stationary node 100 also has a battery 150 for receiving power therefrom.

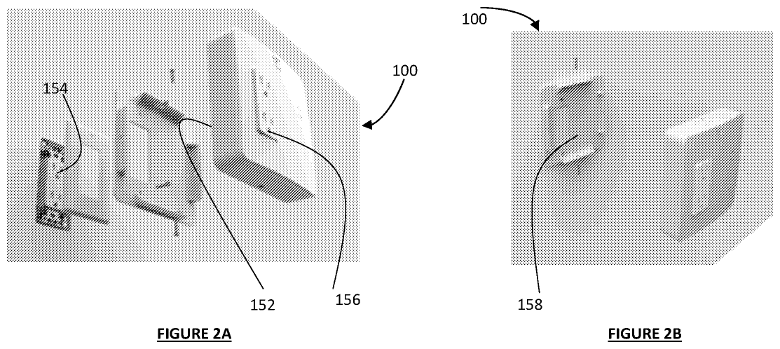

Referring to FIG. 2A, in one embodiment, the wireless stationary node 100 further has a power output interface 152 connectable to a power source 154 of the healthcare facility. The wireless stationary node 100 also has a power input interface 156 adapted to receive an electrical connection from a patient support apparatus 10. The power input interface 156 is electrically connected to the power output interface 152. Such embodiment enables to power the wireless stationary node 100 directly from the power grid.

In the embodiment illustrated in FIG. 2B, the wireless stationary node 100 is fixed on a wall with a socket 158 and is powered with an electrical cable connected to the power grid of the healthcare facility. Such embodiment allows to choose the more convenient position for each wireless stationary node 100 according to the configuration of the patient support apparatus positions in the rooms.

FIG. 3 shows diagrammatically a hospital facility provided with a plurality of rooms and a plurality of wireless stationary nodes 100, each being associated to a patient support apparatus position.

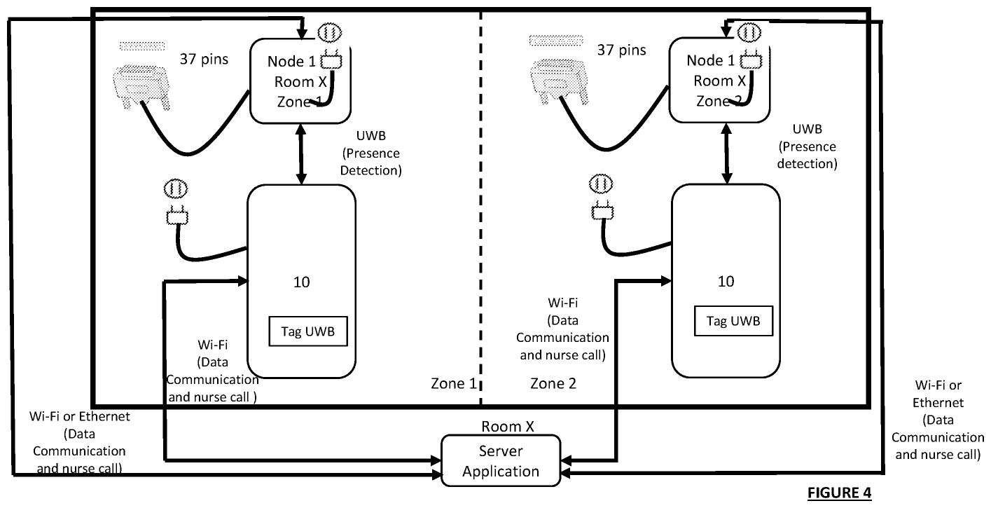

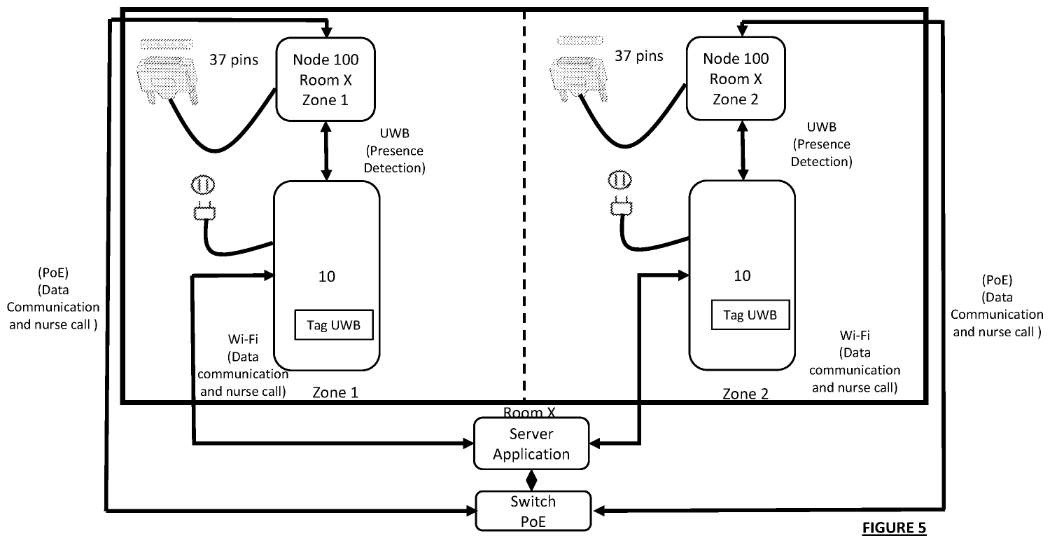

FIG. 4 shows an exemplary system using wireless stationary nodes 100 powered from the hospital power grid while FIG. 5 shows another exemplary system using wireless stationary nodes 100 powered from Power over Ethernet (POE).

Referring again to FIG. 1, in one embodiment, the wireless stationary node 100 has a storage medium (not shown) operatively connected to the processor 102, the storage medium comprising computer-readable instructions. The processor 102, upon executing the computer-readable instructions, is configured to provide the precise location to a remote server 402 embedding a RTLS application for visual representation of the location of the portable identification device, as it will described in more detailed below. In this embodiment, the determination of the location may be performed at the wireless stationary node, i.e. locally without any server.

In a further embodiment, the computer-readable instructions further have configuration instructions enabling to automatically update the wireless stationary node 100 through Over The Air updates. For example, the configuration instructions may implement an automatic check of a most up to date software version available on the remote server 402. If a current software version configurating the wireless stationary node 100 is not the same than the most up to date version, then the processor 102 will download the up to date software version and update the wireless stationary node 100 automatically. The skilled addressee will appreciate that unique identification data associated with a corresponding wireless stationary node 100 may also be configurated remotely. This may be helpful when a wireless stationary node 100 is moved from a patient support location to another.

### Portable Identification Device

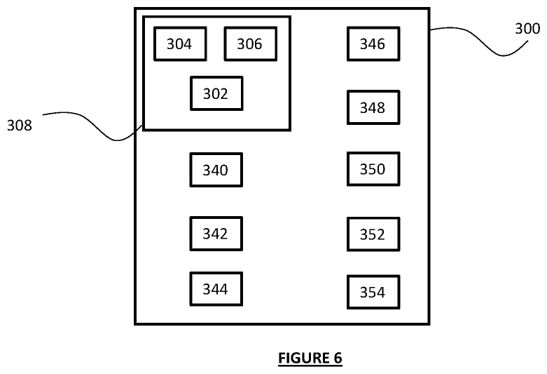

Referring now to FIG. 6, a portable identification device 300 in accordance with one embodiment of a further aspect of the technology will be described. The portable identification device 300 may be used on various medical equipment, medical personnel, or other employees and visitors. The portable identification device 300 has unique identification data associated thereto that are typically broadcasted to adapted receivers available in the vicinity, such as the wireless stationary node 100 previously described for example.

In one embodiment, the portable identification device 300 may be easily installed on a fleet of patient support apparatus 10 of any manufacturer at a low cost. The medical equipment that are specifically associated and used with a specific patient in a room may also be precisely located in the room. This precise location determination would help in the deployment of a complete integrated managing solution for a healthcare facility that is highly reliable while still very cost effective and easy to implement. Knowledge of precise location of medical caregivers, housekeepers, maintenance employees, authorized visitors or any other persons equipped with a portable identification device 300 may also further improve overall efficiency and reliability of all operations related to patient's care management and beds management, as it will become apparent.

The portable identification device 300 has a first transceiver 304 operable to exchange first signals with a first transceiver 104 of a wireless stationary node 100, the exchanged first signals being representative of a precise distance of the portable identification device 300 to the wireless stationary node 100. The portable identification device 300 also has a second transceiver 306 operable to exchange second signals with a second transceiver 106 of the wireless stationary node 100, the exchanged second signals being representative of a precise angle of arrival of the portable identification device 300 to the wireless stationary node 100.

The angle of arrival of the received signal may be determined by any suitable method, such as by determining the phase difference of the received signal at two spaced-apart antennas of the receiving device.

In one embodiment, the first transceiver 304 has a UWB transceiver exchanging UWB signals and the second transceiver 306 has a Bluetooth transceiver exchanging Bluetooth signals. In the following description of exemplary embodiments and as previously mentioned with respect to the wireless stationary node 100, the first transceiver 304 will generally be referred to as the UWB transceiver 304 while the second transceiver 306 will generally be referred to as the Bluetooth transceiver 306 although other types of transceivers may be used. For example, in an alternative embodiment, each of the first transceiver and the second transceiver may have a distinct UWB transceiver. In this example, the determined distance may be determined according to signal strength, time of fly or other known techniques.

In one embodiment, the portable identification device 300 has a processor 302 which may be embedded in a microcontroller which is further provided with at least one non-transitory computer-readable storage medium (not shown). The microcontroller is operatively connected to the UWB transceiver 304, the Bluetooth transceiver 306 and additional components that may be added for a particular application, as detailed below. The microcontroller is configured to communicate with and/or operate the other components of the portable identification device 300.

In one embodiment, the processor 302, the UWB transceiver 304 and the Bluetooth transceiver 306, which define a location module 308, are integrated in a single chip. The single chip has a first and a second integrated antenna, the first antenna being adapted for UWB communication and the second antenna being adapted for Bluetooth communication. In one embodiment, the chip DWM1001C from Decawave previously mentioned may be used. Such chip enables the wireless stationary node to work as a UWB anchor and/or a Bluetooth anchor.

It should be understood that either or both of the first transceiver 204 and the second transceiver 206 could instead be implemented as either a transmitter or a receiver, or as a combination transmitter-receiver. The term “transceiver” as used herein should be understood to encompass these implementations.

In one embodiment, the portable identification device 300 uses only a single transceiver 304 for exchanging signals with the wireless stationary node. In this embodiment, the first and second signals may be of the same type, corresponding to the type of transceiver 304. For example, if the transceiver 304 is a UWB transceiver, the first and second signals are first and second UWB signals. If the transceiver 304 is an ultrasonic transceiver, the first and second signals are first and second ultrasonic signals. In another embodiment, the single transceiver 304 transmits only one signal, for example an UWB signal or an ultrasonic signal, that can be used to determine both the distance to the portable wireless stationary node and the angle of arrival of the signal. The angle of arrival of the received signal may be determined by any suitable method, such as by determining the phase difference of the received signal at two spaced-apart antennas of the receiving device.

The portable identification device 300 also has an event sensor 340 for sensing an event and operable for providing an event signal to the wireless stationary node 100 with one of the UWB transceiver 304 and the Bluetooth transceiver 306. The event sensor 340 may have a push button easily operable by the user. In the case where the portable identification device 300 is used as an identification tag on caregiver, the caregiver may send an alert message rapidly, the message being directly associated with the location of the caregiver and thus with the associated patient. In one embodiment, such event sensor 340 may be implemented as a nurse call button. In such a case, activation of the event sensor 340 will transmit an alert signal or message to the nurse call system for further action. In another embodiment, the event sensor 340 is configured to detect a predetermined event and to provide the event signal based on occurrence of the predetermined event. As a non-limitative example, the portable identification device may be used on a patient. Detecting acceleration associated to the portable identification device and/or a relative height position may be representative of a fall of the patient. Upon detection of a fall event, the event sensor 340 is configured to automatically sent the event signal for further action. In one embodiment, the caregiver located the closest to the patient may receive the event signal informing that the patient needs immediate attention. Various other events may be detected.

Once a connection is established between the portable identification device 300 and a wireless stationary node 100, the portable identification device 300 may optionally communicate data directly to the wireless stationary node 100. The data could be, for example, status information about the medical equipment with which the portable identification device 300 is associated, such as whether the medical equipment is operating correctly. The data could be, for example, sensor results that have been collected by the medical equipment in the course of monitoring the patient. The wireless stationary node 100 may, in some embodiments, transmit the information from the portable identification device 300 to a hospital network 400. In the event that access to the hospital network 400 is interrupted, in some embodiments the wireless stationary node 100 may store the information until network access is restored, at which point the wireless stationary node 100 may transmit the stored data to the hospital network 400. In this way, data that are intended for use or storage within the hospital network 400 can be preserved even when the hospital network 400 is temporarily unavailable.

The connection between the wireless stationary node 100 and the portable identification device 200, 300 may in some embodiments enable one or more functions relating to the person or medical equipment associated with the portable identification device 200, 300. These and similar advantages may be achieved, for example, by knowledge of the locations of individual personnel and medical devices within the hospital, or by the hospital network 400 sending control information to, or receiving information from, individual medical devices via the wireless stationary node to which they are connected. Examples of these functions include:
a. Management, supervision and control of alarms generated by medical equipment in a zone of the hospital. The zone from which an alarm originates may be identified based on the location of the portable identification device 200, 300 associated with the device that generated the alarm. Alarms may be prioritized or otherwise managed based on a zone from which they originate, or associated with a patient who is located in the same zone as the medical equipment that generated the alarm. Medical personnel responding to an alarm could in some embodiments be directed to the precise location of the device that generated the alarm, and could in some embodiments be provided with useful information such as the medical history of the patient associated with the device or the zone from which the alarm originated. In some embodiments, an alarm generated by a piece of medical equipment may alert only nearby medical staff, based on the locations of the portable identification devices 200, 300 associated with the medical staff, thereby improving response times and reducing distractions for medical staff who are not in a position to respond to the alarm.
b. Management, supervision and control of medical equipment in a zone of the hospital. In some embodiments, the location, status, and operation of medical equipment can be known and controlled. Equipment in one area of the hospital that is not currently being used may be relocated to a different area where it is needed. In some embodiments, malfunctioning equipment may be tracked, and efficiently repaired or replaced. In some embodiments, medical equipment that is being used for a particular patient may be automatically associated with that patient based on proximity to the patient, the patient's bed, or the same wireless stationary node with which the patient or the patient's bed is connected.
c. Management, supervision and control of data communicated between medical equipment and a zone of the hospital. In some embodiments, data generated by medical equipment can be associated with a patient based on the location of the medical equipment and the patient. In some embodiments, data generated by medical equipment may be communicated to the hospital network 400 via the wireless stationary node 100, and accessed remotely by medical staff who are responsible for treating the associated patient.
d. Management, supervision and control of activities and services performed by hospital staff in a zone of the hospital. In some embodiments, the locations of hospital staff within the hospital could be known and tracked. This information could be used, for example, to ensure that hospital staff are properly allocated to their designated areas, that all areas of the hospital are properly staffed, or that procedures performed by hospital staff are properly recorded, associated, and invoiced to the correct patient based on the location of the medical staff when the procedures were performed.
e. Management, supervision and control of data communicated between hospital staff and a hospital zone.
f. Management, supervision, and control of access to hospital zones. In some embodiments, the locations of hospital personnel and equipment could be tracked or monitored to prevent unauthorized access to restricted zones of the hospital. In some embodiments, access to certain zones of the hospital might be restricted for safety reasons, for example in a psychiatric ward, a ward where an infectious disease such as Covid-19 is known or suspected to be present, or a ward where patients need specialized care that can only be provided by specially licensed or trained staff. In some embodiments, an alarm or a nurse call system originating from a restricted access zone could alert only those medical staff who are authorized to enter the restricted access zone. In some embodiments, access to zones could be tracked to verify that service or preventive maintenance has been performed on medical equipment, as evidenced by the presence of a maintenance worker in the vicinity of the medical equipment.
g. Management, supervision and control of a patient's activities. A portable identification device 200, 300 may be associated with a patient to track the location of the patient. For example, if a patient is advised or required to stay in bed, an alarm could be generated upon detecting that the patient and the bed are no longer in the same location, or that the patient has been away from his bed for longer than a predetermined amount of time. In another example, if a patient is advised or required to walk a certain amount to aid in recovery, an alarm could be generated if the patient has not spent enough time out of bed, or the distance walked by the patient can be estimated by the recorded locations of the patient over time. In another example, a patient's location could be monitored to ensure that the patient remains only in areas of the hospital to which the patient is permitted access. In another example, a missing patient, for example a patient suffering from Alzheimer's disease who has left his room and is unable to find his way back, could be located and retrieved based on the location of his portable identification device. In another example, it could be determined based on the location of a patient that the patient is out of bed but still within his room, or that the patient has or is about to fall out of bed, or that the patient is in the bathroom.
h. Management, supervision and control of activities and/or services carried out in a zone of the hospital. In some embodiments, people other than medical staff and patients, such as visitors and external consultants, could be monitored to ensure that they remain in authorized areas of the hospital, or that they are no longer present in the hospital at unauthorized times, such as after visiting hours have ended.
i. Management, supervision and control of data communicated between the subject and a zone of the hospital.
j. Monitoring and management of alarms based on the locations of medical staff. In some embodiments, if a doctor or nurse is known to be in the same zone as a patient, an alarm associated with a patient may be suppressed, or sent only to the doctor or nurse who is already attending to the patient. For example, if a patient is being monitored to make sure the patient remains in bed, the presence of hospital staff near the patient may indicate that the patient is observed to be out of bed as a result of the patient receiving some treatment and moving within the bed, being bathed or having his bedding changed, or getting out of bed under medical supervision. In this example, it may be the case that no alarm is needed because the patient is already being monitored by medical staff.
k. Medical personnel may automatically receive access to a patient's medical records or other relevant information on a smartphone or other wireless device when the medical personnel is in proximity to a patient or a patient's bed.
l. A patient's bed or other medical equipment may permit restricted functions to be performed, such as control or maintenance functions, when it is determined that an authorized maintenance worker is in the same zone as the bed.

In one embodiment, the portable identification device 300 may be installed on any patient support apparatus 10 of any manufacturer to collect location information, as previously described. In such a case, the event sensor 340 may be implemented to provide an easy access to the patient to thereby offer a nurse call button to the patient installed in a bed devoid of any nurse call button.

In one embodiment, it may be determined based on the location or change in location or movement pattern of a portable identification device 200, 300 that the associated bed or medical equipment is being deliberately removed from the room in which it was located. This determination can also or instead be made based on status information transmitted from the bed to the wireless stationary node 100, such as the brakes on the bed being disengaged, or the bed being unplugged from a wall outlet, before being moved. In this case, the portable identification device 200, 300 may be disconnected from the wireless stationary node 100 in a deliberate or controlled manner, and any alarm associated with the equipment being disconnected may be suppressed or cancelled.

In some embodiments, the portable identification device 300 may be provided with additional modules that may be used according to various combinations depending of a specific application.

In some embodiments, the portable identification device 300 has a lithium battery 342 to provide power thereto and a USB C port 344 enabling to configurate and update the portable identification device 300 from a PC or mobile application for example. The portable identification device 300 may also be provided with a power managing module 346 to manage the battery 342 and the power provided thereto. In some embodiments, the portable identification device 300 has a UART (Universal Asynchronous Receiver Transmitter) 348 and a programmer 350 between the USB C port 344 and the UART 348. In some embodiments, the portable identification device 300 has an NFC sensor 352 for enabling wireless configuration thereof. In some embodiments, the portable identification device 300 also has an induction charging station 354 for wirelessly recharging the portable identification device 300.

Patient Support Apparatus with Identification Device

As it should now become apparent, in one embodiment and according to a further aspect of the technology, the portable identification device 300 previously described may be used on a patient support apparatus 10 for precise geolocation purpose.

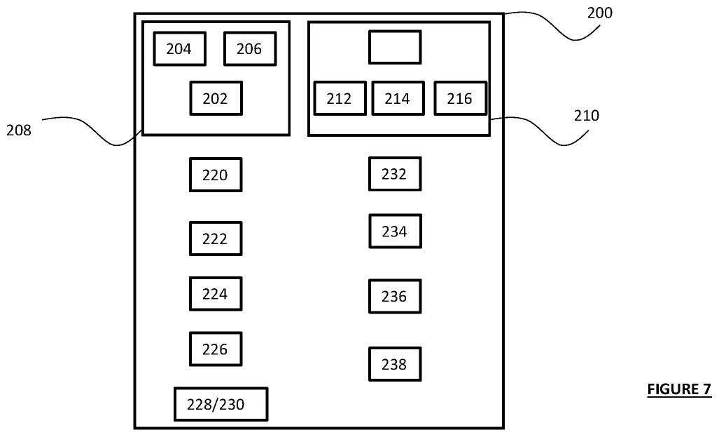

Accordingly, and referring now to FIGS. 1 and 7, there is provided a patient support apparatus 10 having a support surface for supporting a patient. The patient support apparatus 10 has a first transceiver 204 operable to exchange first signals with a first transceiver 104 of a wireless stationary node 100, the exchanged first signals being representative of a precise distance of the patient support apparatus 10 to the wireless stationary node 100. The patient support apparatus 10 further has a second transceiver 206 operable to exchange second signals with a second transceiver 106 of the wireless stationary node 100, the exchanged second signals being representative of a precise angle of arrival of the patient support apparatus 10 to the wireless stationary node 100. As it should be appreciated, in one embodiment, the patient support apparatus 10 is also provided with a processor 202 operatively connected to the first transceiver 204 and to the second transceiver 206. The processor 202, the first transceiver 204 and the second transceiver 206 will be referred as the location module 208 thereinafter.

The angle of arrival of the received signal may be determined by any suitable method, such as by determining the phase difference of the received signal at two spaced-apart antennas of the receiving device.

In one embodiment, the first transceiver 204 has a UWB transceiver exchanging UWB signals and the second transceiver 206 has a Bluetooth transceiver exchanging Bluetooth signals. In the following description of exemplary embodiments and as previously mentioned, the first transceiver 204 will generally be referred to as the UWB transceiver 204 while the second transceiver 206 will generally be referred to as the Bluetooth transceiver 206 although other types of transceivers may be used.

In one embodiment, the processor 202, the UWB transceiver 204 and the Bluetooth transceiver 206 are integrated in a single chip. The single chip has a first and a second integrated antenna, the first antenna being adapted for UWB communication and the second antenna being adapted for Bluetooth communication. In one embodiment, a chip DWM1001C from Decawave, Ireland, may be used. Such chip may enable the location module 208 of the patient support apparatus 10 to work as a UWB anchor and/or a Bluetooth anchor, as it should be apparent to the skilled addressee.

It should be understood that either or both of the first transceiver 204 and the second transceiver 206 could instead be implemented as either a transmitter or a receiver, or as a combination transmitter-receiver. The term “transceiver” as used herein should be understood to encompass these implementations.

In one embodiment, the portable identification device 200 uses only a single transceiver 204 for exchanging signals with the wireless stationary node. In this embodiment, the first and second signals may be of the same type, corresponding to the type of transceiver 204. For example, if the transceiver 204 is a UWB transceiver, the first and second signals are first and second UWB signals. If the transceiver 204 is an ultrasonic transceiver, the first and second signals are first and second ultrasonic signals. In another embodiment, the single transceiver 204 transmits only one signal, for example an UWB signal or an ultrasonic signal, that can be used to determine both the distance to the portable wireless stationary node and the angle of arrival of the signal. The angle of arrival of the received signal may be determined by any suitable method, such as by determining the phase difference of the received signal at two spaced-apart antennas of the receiving device.

The patient support apparatus 10 is also provided with a communication module 210 for wirelessly connecting the patient support apparatus 10 to a facility network 400 and/or to the wireless stationary node 100. The communication module 210 has at least one of a Wi-Fi transceiver 212, a UWB transceiver (not shown), a Bluetooth transceiver 214 and an ethernet interface (not shown), the communication module 210 being adapted to exchange data with a communication module 110 of the wireless stationary node 100, as previously described. As previously mentioned, data may be exchanged bidirectionally through the Wi-Fi transceivers, the UWB transceivers or the Ethernet connection. The patient support apparatus 10 also has a controller 216 adapted to automatically establish a communication link between the communication module 210 of the patient support apparatus 10 and the communication module 110 of the wireless stationary node 100 without requiring a user to select or identify the wireless stationary node 100. In one embodiment, the communication module 210 of the patient support apparatus 10 has a chip ESP32 from Espressif Systems. This chip enables Wi-Fi/Ethernet/BLE (Bluetooth Low Energy) communication with a single integrated antenna for WI-Fi/BLE communication.

In one embodiment, the processor 202 of the location module 208 is configured to convert UART messages on the LIN (Local Interconnected Network) connecting the location module 208 and the communication module 210. In a further embodiment, the processor 202 of the location module 208 is further configured to communicate with the processor 216 of the communication module 210 and enables configuration, data transfer and updates associated with each module 208, 210.

In an embodiment where the patient support apparatus 10 is not provided with a communication module 210, the chip chosen for the location module 208 could be, instead of the DWM1001C from Decawave, a chip ESP32 from Espressif Systems. This arrangement would provide a Wi-Fi connection to the patient support apparatus 10 even without the communication module 210.

In some embodiments, the patient support apparatus 10 may be provided with additional modules that may be used according to various combinations depending of a specific application. For example, the modules previously described in reference to the portable identification device 300 may be similarly used in combination with the location module 208 of the patient support apparatus 10.

In some embodiments, the patient support apparatus 10 has an AC/DC power port 220 for powering directly the location module 208, the communication module 210 and the additional modules from the power grid through the powered patient support apparatus 10. In alternate embodiments, the location module 208 may be provided with an independent lithium battery 222. In other embodiments, the location module 208 and other modules are powered from main batteries provided on the patient support apparatus 10. In some embodiments, a power and communication port LIN/CAN 224 is provided for powering the location module 208 directly from the main batteries of the patient support apparatus 10 or from the communication module 210 thereof. The LIN/CAN port 224 can also be used for communication between the location module 208 and the communication module 210. In some embodiments, the location module 208 of the patient support apparatus 10 is further provided with a power managing module 226 to manage the battery 222 and the power provided to the locating module 208 and associated modules.

The skilled addressee will appreciate that thanks to the independent battery 222 and/or the main batteries, the patient support apparatus 10 may still be located, at least approximatively, even when disconnected from the main power grid. For example, the patient support apparatus 10 can be located during displacement thereof, thanks to the wireless stationary nodes 100 conveniently distributed in the hospital. In one embodiment, the hallways of the hospital are not provided with wireless stationary nodes 100 since the wireless stationary nodes 100 of the adjacent rooms may be used to locate a moving patient support apparatus in the vicinity thereof. Geofencing technologies may further be used to collect additional data related to the relative position of a moving patient support apparatus 10.

The skilled addressee will also appreciate that bidirectional data exchange may still be active during the displacement of the patient apparatus. This may be of great advantage in ICU units since vital signs monitored with portable monitors accompanying the patient during his displacement may still be also monitored remotely and/or store, thanks to the data exchange.

In some embodiments, the precise location determination capabilities of the wireless stationary node 100 previously described may be implemented in the patient support apparatus 10 provided with a location module 208. Accordingly, in one embodiment, the patient support apparatus 10 has a processor 202 operatively connected to the UWB transceiver 204 and the Bluetooth transceiver 206. The processor 202 is adapted to determine a precise distance of the patient support apparatus 10 from the wireless stationary node 100 based on the exchanged UWB signals. The processor 202 is further adapted to determine a precise angle of arrival of the exchanged Bluetooth signals. Based on the fixed position of the wireless stationary node 100 that is first transmitted to the location module 208 of the patient support apparatus 10, the processor 202 is further configured for determining a precise location of the patient support apparatus 10 based on the determined precise distance and the determined precise angle of arrival. The precise determined location may then be transmitted to the wireless stationary node 10 or to a remote server 402 through the hospital network 400.

The angle of arrival of the received signal may be determined by any suitable method, such as by determining the phase difference of the received signal at two spaced-apart antennas of the receiving device.

In some embodiments, the patient support apparatus 10 is provided with a storage medium operatively connected to the processor 102, the storage medium comprising computer-readable instructions, the processor 102, upon executing the computer-readable instructions, being configured to provide the precise location to a remote server 402 embedding a RTLS application 404 for visual representation of the location of the patient support apparatus 10.

In some embodiments, the patient support apparatus 10 further has one of an infrared module 228 and an ultrasound module 230 for detecting only a wireless stationary node 100 that is located in a same room in which the patient support apparatus 10 is installed to enable implementation of geofencing capabilities.

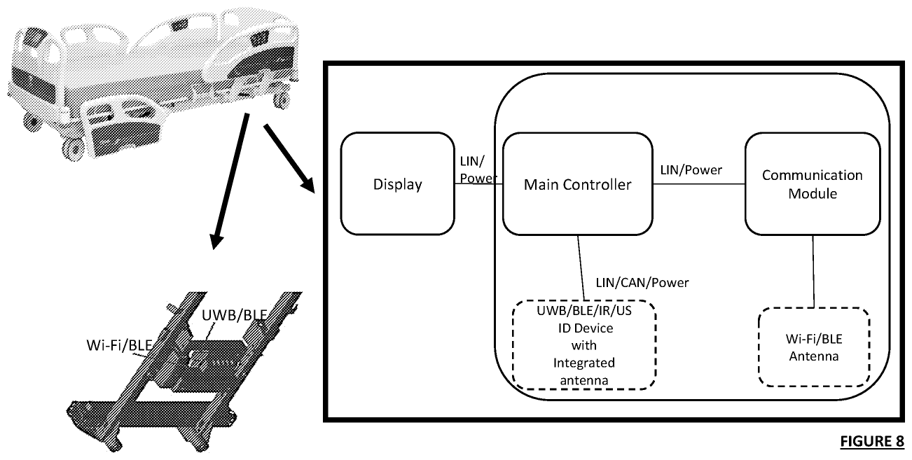

In further embodiments, as it should now be apparent to the skilled addressee, the location module 208 of the patient support apparatus 10 may be further configured to communicate with the portable identification devices carried by employees. The location module 208 may provide the main controller (see FIG. 8) of the patient support apparatus 10 with identification data of the detected employees. Based on recognition of the authorization level of the detected portable identification device, the main controller of the patient support apparatus 10 may cancel alert calls or bed alarms, and also activate/deactivate some functions of the patient support apparatus 10 and/or access thereof. Alternatively, the recognition of the authorization level of the detected portable identification devices may be implemented within the wireless stationary node 100 and then communicated to the main controller of the patient support apparatus 10.

FIG. 7 shows a communication module 210 of a patient support apparatus 10, according to an exemplary embodiment. The communication module 210 has a UWB/BLE/Ethernet chip, for example, the ESP32 chip previously described. The communication module 210 is also provided with a RJ45 port enabling Ethernet communication with the hospital network 400. The communication module 210 also has a nurse call interface 232 comprising a connector such as a 37 pins connector for connecting to an existing nurse call system of the healthcare facility and nurse call relays 234 configured to transfer nurse calls to the nurse call system of the healthcare facility. The communication module 210 is also provided with a USB-hub circuit 236 enabling to program the UWB transceiver and the Bluetooth transceiver independently from each other. The communication module 210 also has a USB B port 238 enabling to configurate and update the communication module 210 and other modules such as the location module 208, for example from a PC or mobile application.

FIG. 8 shows a patient support apparatus having a control box mounted to the frame thereof. The control box embeds the main controller of the patient support apparatus and a communication module operatively connected thereto. As it should be noted, in the illustrated embodiment, the modules provided with antenna are mounted below the control box.

The skilled addressee will appreciate that the mattress used on the patient support apparatus 10 may, alternatively or in addition to the patient support apparatus 10, also be provided with the previously described identification device.

### Location Determination System

As it should now be apparent to the skilled addressee, the wireless node 100, the portable identification devices 200, 300 and the patient support apparatus 10 previously described can advantageously be used in combination to provide a location determination system for determining location of a plurality of portable identification devices in a healthcare facility having a plurality of rooms, according to a further aspect of the technology.

Referring again to FIG. 1, in one embodiment, the location determination system 500 is provided with a plurality of wireless stationary nodes 100 based on UWB/BLE technology, for example the ones previously described, or alternate embodiments described above. Each of the wireless stationary nodes 100 has a unique identifier and the plurality of wireless stationary nodes 100 are distributed in the rooms of the facility. The precise location of each of the identification devices 200, 300 is determined using a single one of the wireless stationary nodes 100 closest to the identification device 200, 300 in the facility. The single one of the wireless stationary nodes 100 that is used is a wireless stationary node 100 that is in the same room than the portable identification device 200, 300 for which precise location is determined.

The location determination system 500 is also provided with a remote server 402 operatively connected to the wireless stationary nodes 100 and operable to receive the respective location of each of the identification devices 200, 300. The remote server 402 embeds a real-time locating system (RTLS) application 404 that is configured for visual representation of the location of each of the identification devices 200, 300 in the facility, as further detailed below.

In one embodiment, the facility comprises a plurality of patient support apparatus positions, each of the patient support apparatus positions being equipped with a single one of the plurality of the wireless stationary nodes 100.

The skilled addressee will appreciate that various elements previously described may be combined together while some may be omitted to tailor a location determination system 500 according to specific needs.

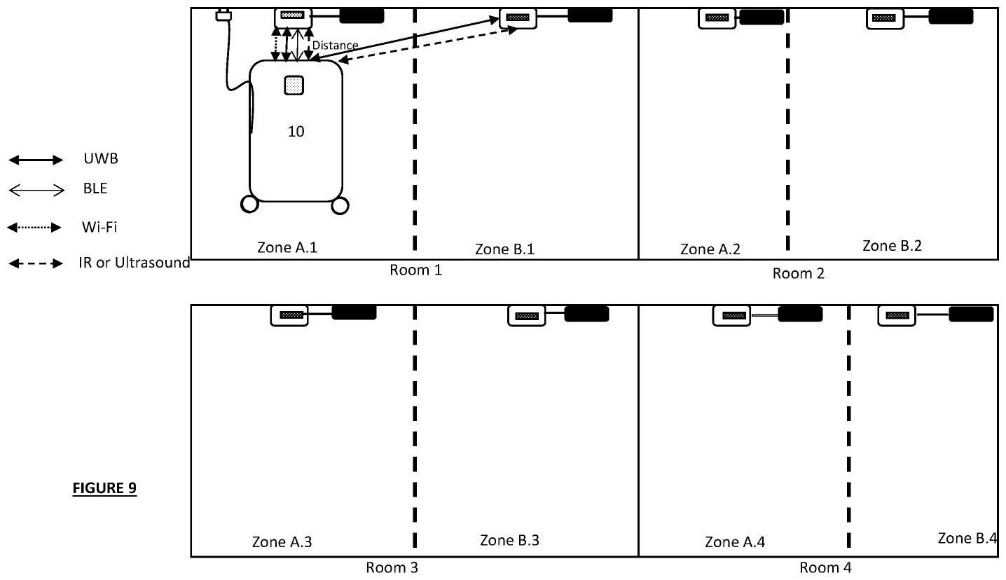

Referring to FIG. 9, there is shown a system 600 that may be used to wirelessly connect a patient support apparatus 10 on an existing nurse call system.

Accordingly, the patient support apparatus 10 is first positioned in the destination room in the vicinity of the wireless stationary node 100 and powered through the power grid. At this point, various alternatives may be considered. According to a first alternative, the infrared module 228 or the ultrasound module 230 of the patient support apparatus 10 is first activated for detecting only the wireless stationary nodes 100 that are located in the same room of the patient support apparatus 10. For that purpose, each of the wireless stationary nodes 100 located in the same room has a device ID that is a common device ID to all the stationary nodes 100 of the same room, which is distinct from another common device ID used in an adjacent room. Then, the UWB chip of the patient support apparatus 10 is activated to detect the wireless stationary node 100 in the same room that is the closest to the patient support apparatus 10. According to a second alternative, the UWB chip of the wireless stationary node 100 that is provided with two integrated antennas automatically detects the patient support apparatus 10 that is the closest thereto. With the determination of the angle of arrival, a patient support apparatus 10 located less than 1.5 meter from the wireless stationary node 100 is detected. According to a third alternative, the infrared module 134 of the wireless stationary node 100 and the UWB chip of the wireless stationary node 100 provided with a single antenna are used in combination to detect the closest patient support apparatus 10 in the same room that is at a distance of less than 1.5 meter from the wireless stationary node 100.

The angle of arrival of the received signal may be determined by any suitable method, such as by determining the phase difference of the received signal at two spaced-apart antennas of the receiving device.

At that point, the patient support apparatus 10 is connected to the nurse call system associated with the wireless stationary node 100 with the UWB module, the Bluetooth module or the Wi-Fi module. The patient support apparatus 10 is configured to store automatically his position and can also display this position at a display screen. Visual or audible signals may be provided to the user when the wireless stationary node 100 and the patient support apparatus 10 are connected together. At that point, the wireless stationary node 100 takes a connected status and prevent another patient support apparatus 10 to attempt to connect on the same nurse call port.

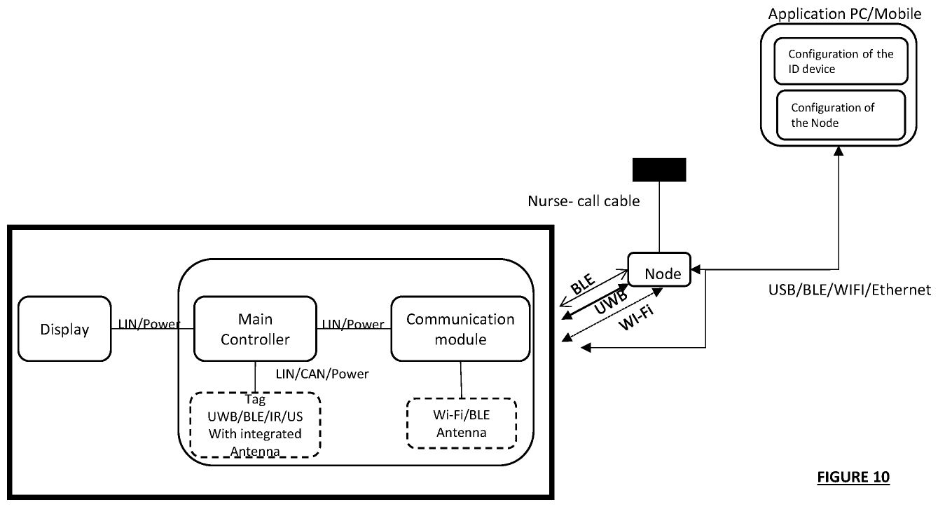

In this embodiment, as illustrated in FIG. 10, a software PC application or mobile application 404 may be used to configure the wireless stationary node 100 and the portable identification device 200 or 300 mounted onto the patient support apparatus 10. The PC or mobile application 404 may be used to provide identification data to the wireless stationary node 100, for example a name representative of its position in the hospital, and also to provide identification data to the patient support apparatus 10, for example a name representative of his associated position in the hospital. The application 404 may also be used for software updates of both the wireless stationary node 100 and the patient support apparatus 10. The application 404 may also be used for configurating a selection of the UWB signals or the Bluetooth signals for the communication of the communication modules 210, 110 of the patient support apparatus 10 and the wireless stationary node 100. The application also enables to configure the refresh rate of the position of the patient support apparatus 10 in the application 404.

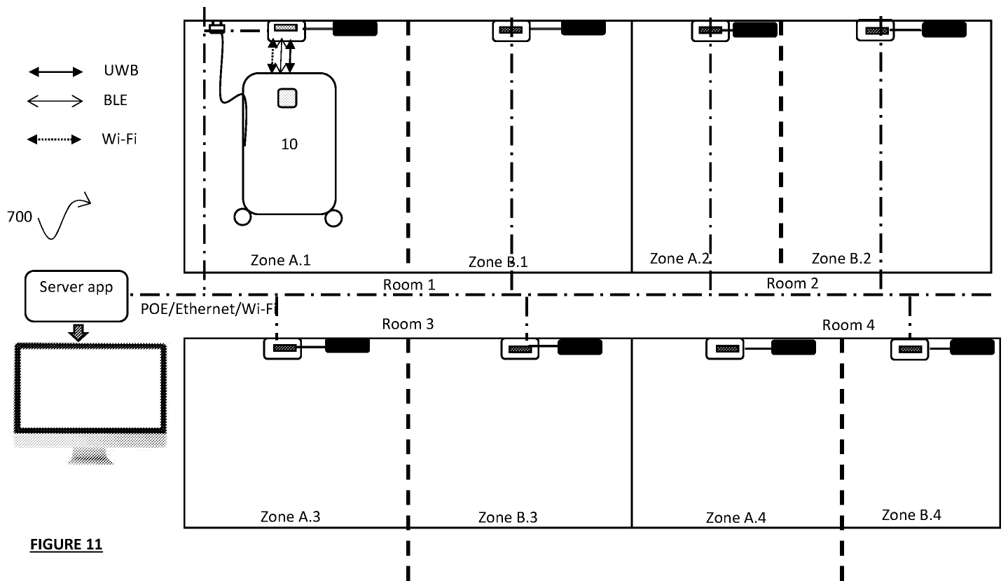

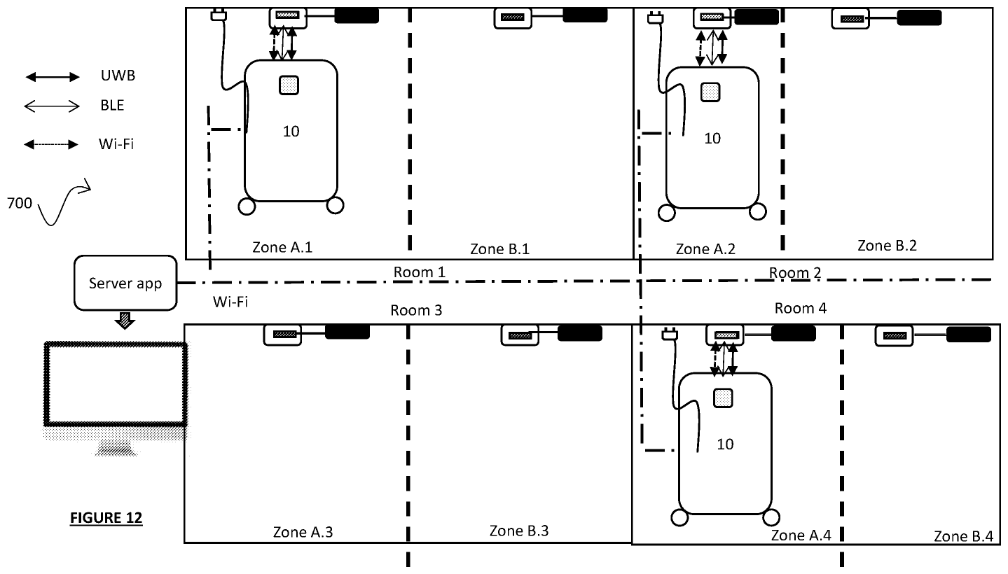

Referring to FIGS. 11 and 12, there is shown a location determination system 700 that may be used to wirelessly and automatically exchange data between the patient support apparatus 10 and a web application 404 embedded on a remote server 402 if the patient support apparatus 10 is connected to the nurse call system through the wireless stationary node 100. Data may be exchanged bidirectionally. Thus, once the patient support apparatus 10 has been connected to the nurse call system associated with the wireless stationary node 100, the determined position of the patient support apparatus 10 may be transmitted to the web application 404. In one embodiment, as illustrated in FIG. 11, the patient support apparatus 10 directly transmits his determined position to the web application 404 through the Wi-Fi connection. In an alternate embodiment, as illustrated in FIG. 12, the wireless stationary node 100 transmits the determined position of the patient support apparatus 10 to the web application 404 through the Wi-Fi connection or the Ethernet connection. With this system, various data may be exchanged, for example, in addition to the current position of the patient support apparatus, weight of the patient, bed exit alarms and other data related to the patient and/or the patient support apparatus may be transmitted to the web application 404. The web application 404 may also transmits data to the patient support apparatus, for example, the bed exit alarms may be activated, suspended or modified remotely from the web application 404 without requiring a caregiver to be physically present. As another example, the backseat movement of the patient support apparatus may be limited to a specific range, for example not above 30 degrees.

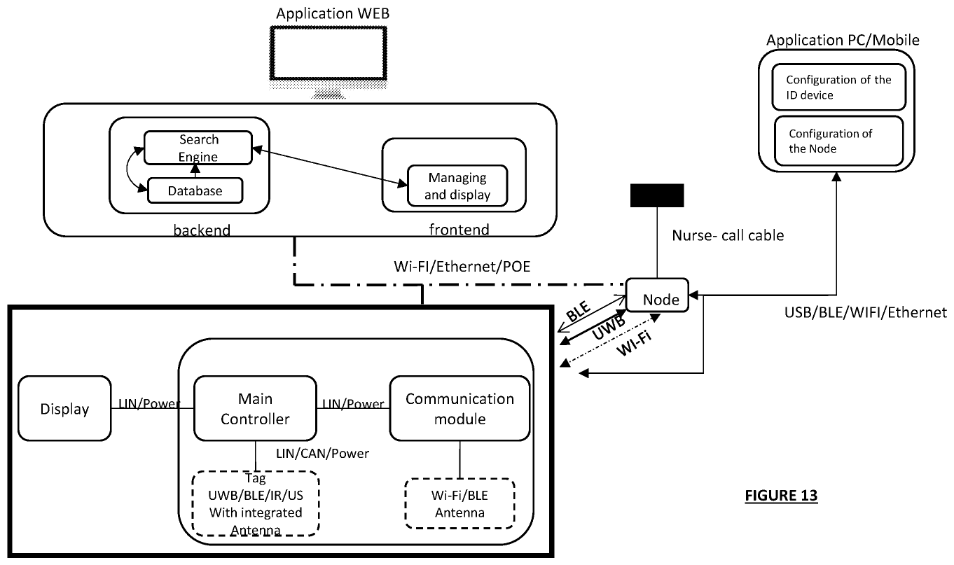

In this embodiment, as illustrated in FIG. 13 and similarly to the embodiment shown in FIG. 10, a software PC application or mobile application 404 may be used to configure the wireless stationary node 100 and the portable identification device 200 or 300 mounted onto the patient support apparatus 10. The PC or mobile application 404 may be used to provide identification data to the wireless stationary node 100, for example a name representative of its position in the hospital, and also to provide identification data to the patient support apparatus 10, for example a name representative of its associated position in the hospital. The application 404 may also be used for software updates of both the wireless stationary node 100 and the patient support apparatus 10. The application 404 may also be used for configurating a selection of the UWB signals or the Bluetooth signals for the communication of the communication modules 210, 110 of the patient support apparatus 10 and the wireless stationary node 100. The application also enables to configure the refresh rate of the position of the patient support apparatus 10 in the application 404.

In this embodiment, the web application 404 has a backend embedding a database and a patient support apparatus position search engine. The database is configured to store the current position of each of the patient support apparatus 10 and the history of each displacement and/or past positions of each patient support apparatus 10. The search engine is configured to enable various searches enabling to locate a particular patient support apparatus 10 according to specific criteria. Indeed, any piece of information that is used as identification data may be used as a search criterion. For example, the web application 404 may allow to locate all the patient support apparatus 10 in a specific building or on a specific level, all the patient support apparatus 10 of a specific manufacturer or even all the patient support apparatus 10 that need a maintenance procedure.

The web application 404 also has a frontend for providing a managing menu to the user. The managing menu may be used to perform specific searches according to various parameters. The front end also has a display section enabling to display all the detected patient support apparatus and their current position, as it should now be apparent.

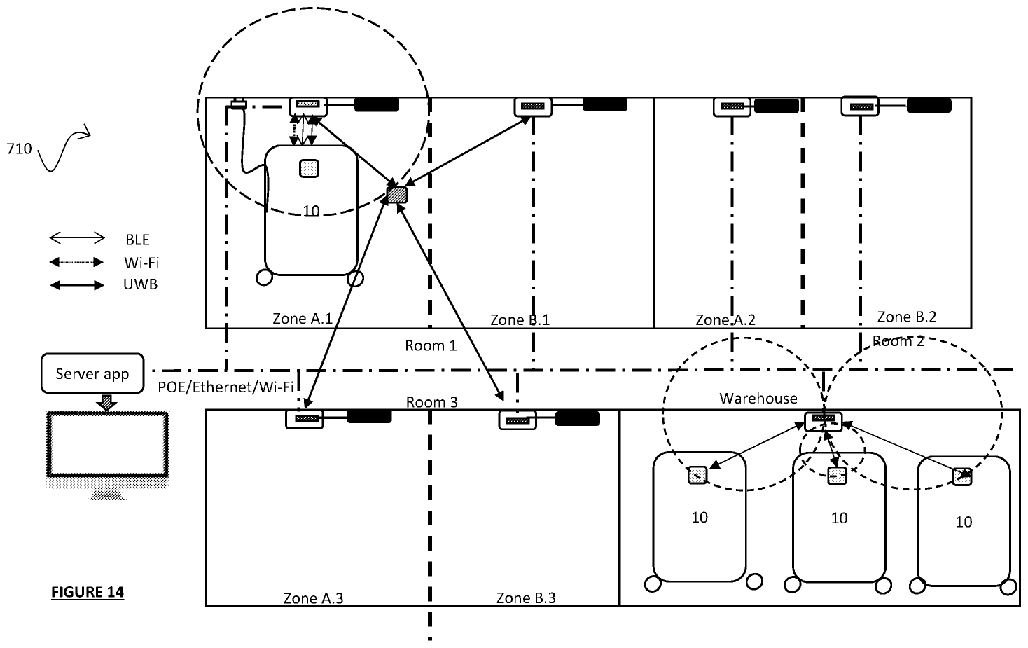

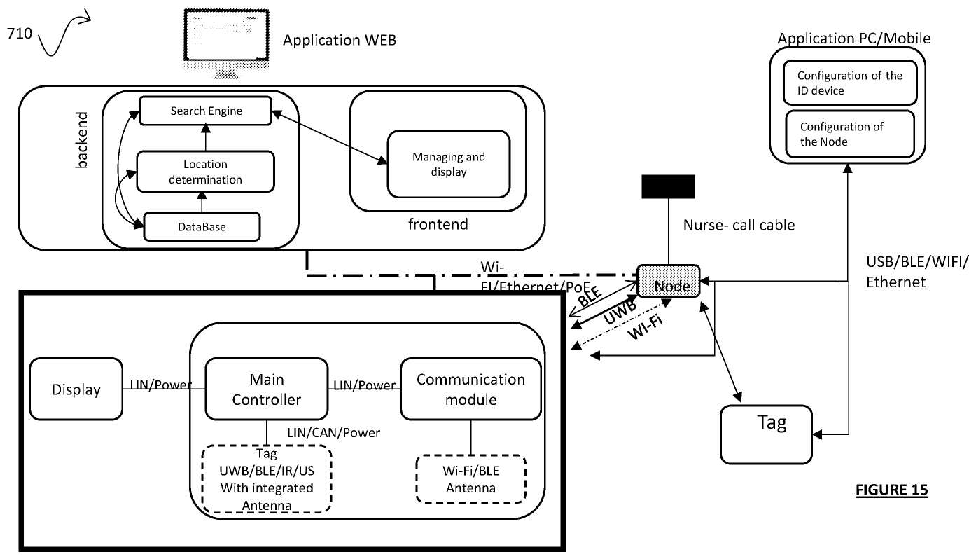

Referring now to FIGS. 14 and 15, there is shown a system 710 that may be used to provide an approximate estimation of the position of hospital personnel, visitors, patients and various medical equipment that may be associated to a patient such as pumps, cardiac monitors or other monitoring devices. This embodiment enables to provide an estimation of the current monitored position even if the monitored device is not connected to a wireless stationary node and/or the nurse call system. As it should be apparent, such functionality is of great advantage since it enables to follow patient support apparatus not connected to the power grid, for example the patient support apparatus that are store in a warehouse for further use or for maintenance procedure. The estimation of the position is determined only in using the determined distance between the corresponding wireless stationary nodes in the vicinity and the corresponding identification device, using the exchanged UWB signals and/or the Bluetooth signals, as detailed above. Once each wireless stationary node in the vicinity of the portable identification device has estimated a position, such position is transmitted from the wireless stationary node to the server of the web application. The application is then configured to determine the closest wireless stationary node to the identification device to thereby provide the approximative position of the identification device.

In this embodiment, as illustrated in FIG. 15 and similarly to the embodiments previously described, a software PC application or mobile application 404 may be used to configure the wireless stationary node 100 and the portable identification device 200 or 300 mounted onto the patient support apparatus 10. The backend of the web application 404 has a database and a patient support apparatus position search engine as previously described. The database is similar to the one previously described but is further provided with a location determination function configured to store the determined distances between each of the wireless stationary nodes 100 and each of the identification devices. The frontend of the web application is further configured for displaying the estimated positions of the identification devices.

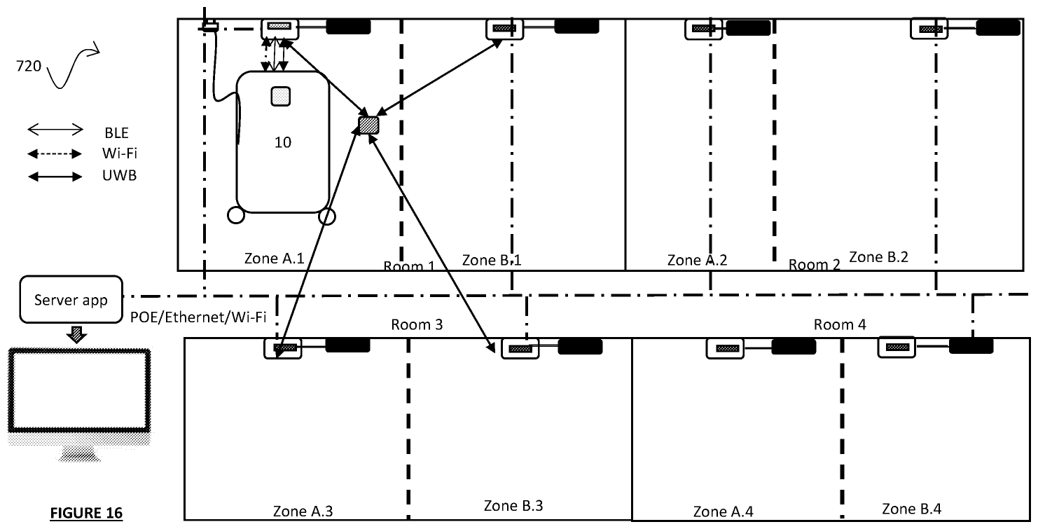

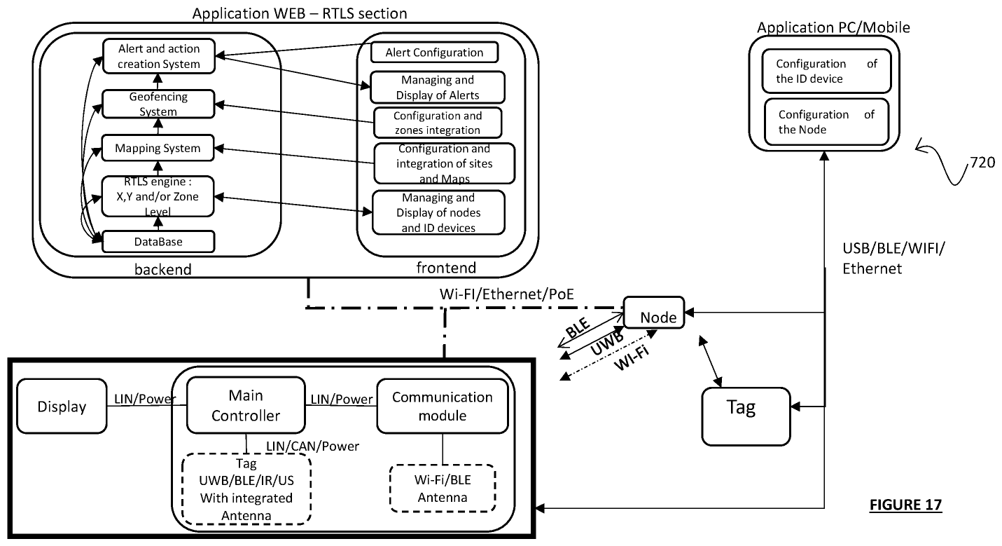

Referring to FIGS. 16 and 17, there is shown a system 720 that may be used to provide a precise position (up to 1 centimeter, in 3 dimensions including height) of hospital personnel, visitors, patients and various medical equipment that may be associated to a patient such as pumps, cardiac monitors and also disconnected patient support apparatus.

The illustrated system 720 may also be used to manage, monitor and/or diagnose actions and alerts of medical equipment and/or personnel equipped with an identification portable device. In one embodiment, the system 720 may further be configured to create specific alerts associated with equipment and/or patients, visitors and employees. This enables a tailored managing of equipment and people for improved monitoring.

In this embodiment, as illustrated in FIG. 17 and similarly to the embodiments previously described, a software PC application or mobile application 404 may be used to configure the wireless stationary node 100 and the portable identification device mounted onto the patient support apparatus 10. The backend of the web application 404 has a database and a patient support apparatus position search engine as previously described. The database is similar to the one previously described but is further provided with a location determination function configured to store the determined distances between each of the wireless stationary nodes 100 and each of the identification devices.

More specifically, in one embodiment, the database is configured to: store the position of each of the wireless stationary nodes, per buildings, levels, rooms, etc.; store the current distance of each corresponding identification device with respect to the four more closest wireless stationary nodes: store the current distance of each corresponding identification device with respect to at least three more closest wireless stationary nodes: store the current position of each of the patient support apparatus per building/level/zone/room; and store the history of each displacement and/or past positions of each patient support apparatus 10 per building/level/zone/room.

In this embodiment, the search engine is a RTLS engine configured to calculate actual positions of the identification devices with a triangulation method with respect to the four more closest wireless stationary node at the maximum and with respect to the three more closest wireless stationary node at the minimum.

The backend of the web application 404 also has a mapping system enabling to integrate the specific map of the hospital in order to associate the determined positions of the identification devices with the real configuration of the hospital. The backend of the web application 404 also has a geofencing system configured to determine a surface associated with a specific zone/room/building. Geofencing technologies previously described may be used. The backend of the web application 404 also has an alert and action creation system IFTT (If This Then That) enabling to program a predetermined response to a predetermined event. This may be of great advantage to further tailor the managing system according to specific needs.

The frontend of the web application is similar to the ones previously described and has further capabilities for integrating hospital configuration maps. The frontend also has further capabilities for enabling the configuration of alert and the display thereof.

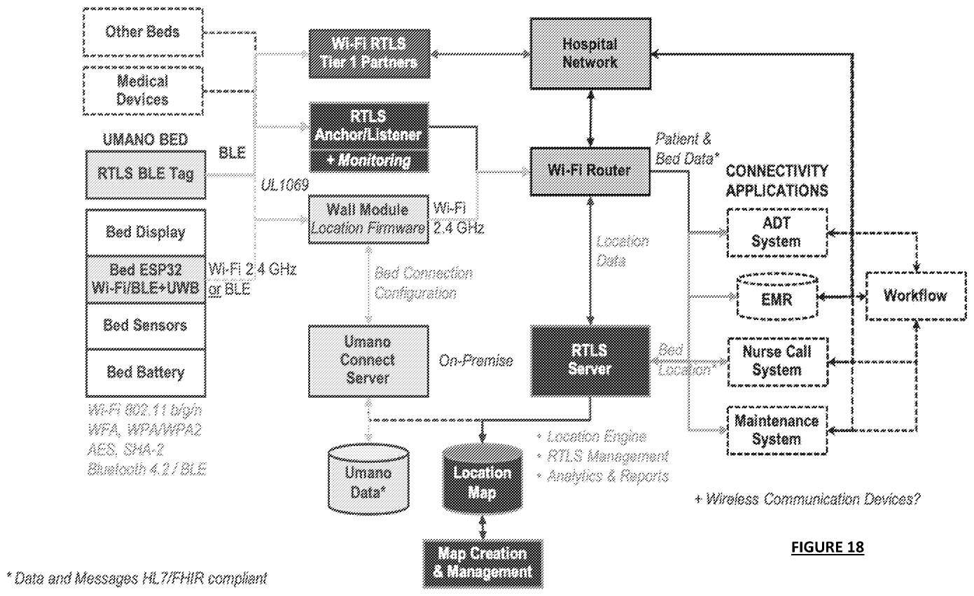

Referring now to FIG. 18, there is shown a managing system configured to exchange wirelessly and automatically bidirectional data between the medical equipment and various computer systems of the hospital such as the ADT system, the EMR system, the nurse call system, the maintenance system, the workflow system, the RTLS system etc., and the patient support apparatus and associated web/mobile applications.

As it should now be apparent to the skilled addressee, the proposed system may thus be easily integrated with the existing systems of various hospitals even in the case the patient support apparatus are from various manufacturers.

For example, in one embodiment, thanks to the determined distances and/or positions, each of the corresponding portable identification devices associated with a corresponding patient may be paired together with high reliability.

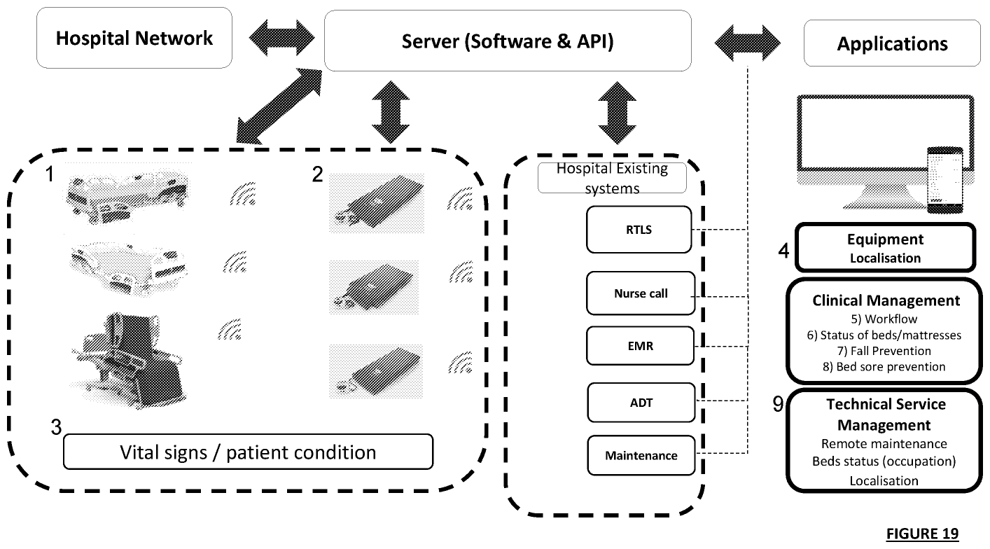

Referring now to FIG. 19, there is shown another managing system similar to the one described with reference to FIG. 18, in accordance with one embodiment. The data that may be exchanged between the server, associated existing hospital systems and associated web or mobile applications may comprise:

For a patient support apparatus and the associated patient, his location, type of bed, height, width, overall position (angle for each section), bed status (brake & Side rail positions for example), bed exit parameters such as the status and the chosen zone detection, nursecall, information displayed on screen, the patient Center of mass, the patient weight, the patient presence in the bed, status of the locks, passwords, various settings (load cell value, . . . ), operating hours, numbers of cycles for each of the actuators, status of brakes or specific mechanical systems, electronical components value, etc. . . . .

For a patient support surface, integrated or independent of the patient support apparatus, and the associated patient, his location, type of surface, surface status, real time operating pressure for each zone, therapy status (ON/OFF), turn Reminder, system value, air leakage, and controller status (alarm, error code . . . ) as non-limitative examples.

In one embodiment, information related to the patient condition may also be exchanged. Such information/data may be related to vital signs monitoring implemented with adapted sensors. Such data may comprise ECG, hearth rate, respiratory rate, oxygen saturation, temperature, glycemia, arterial pressure, sleep apnea. Such information/data may also be related to moisture measurement and detection, urine detection, emotion detection or pain detection as non-limitative examples.

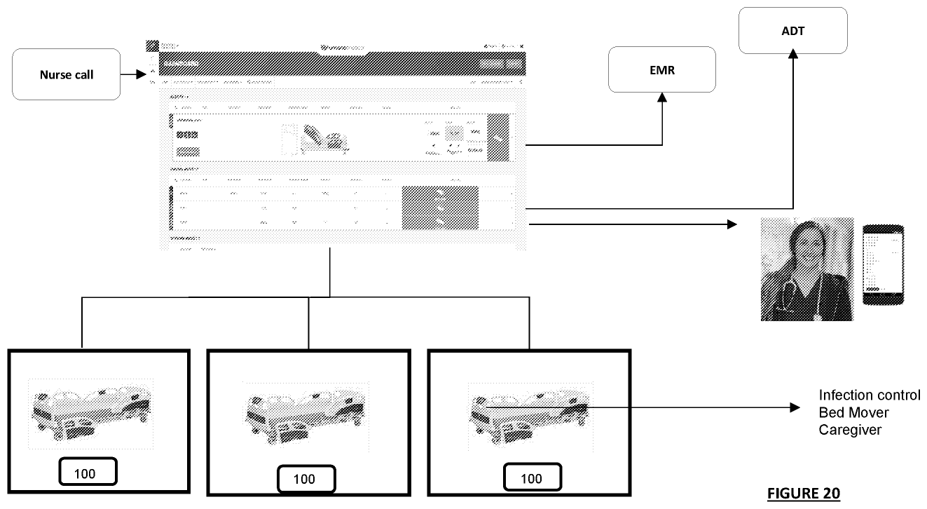

In one embodiment, the workflow of the hospital may also be managed with the managing system. This may be of great advantage to improve overall efficiency of an overall healthcare facility since it can enable, for example, minimization of the time the patient support apparatus is not available between two subsequent uses. Once the patient is authorized to leave the hospital, the information may be given to the managing system that would then communicate with the ADT system. The subsequent workflow may be entirely automated. For example, personnel devised to the cleaning may be informed in real time that the room has now to be reprocessed. A workflow mobile application may be used for that purpose, as illustrated in FIG. 20. Accounting system may also be informed. This accounting system may be provided with every piece of information collected during the stay of the patient so that billing is automatically calculated accordingly.

The skilled addressee will also appreciate that various other monitoring may be implemented. For example, in one embodiment, the managing system is used for fall prevention. Indeed, while the system is able to monitor the center of mass of the patient, the system is further configured to determine a level of agitation for a specific patient, based on the center of mass data. According to specific known behaviors, a fall prediction algorithm may be implemented, for example with an Artificial Intelligence algorithm. If a fall prediction is confirmed, an alert may be sent to the mobile application of caregivers. Alternatively or in addition, an audio message may be send to the patient. Various alerts may be configured, as it should now be apparent.

In a further embodiment wherein the patient support apparatus is provided with a configurable mattress, pressure ulcer and bed sores management may also be implemented and remotely monitored. Actions may then be remotely sent to the patient support apparatus to reduce pressure ulcer and bed sores. In one embodiment, the peak pressure of the mattress may be monitored and alerts may be set. Pressure of various sections of the mattress may also be changed remotely. A Turn reminder may also be implemented. The patient position may be monitored in real time and alerts can be set. Moisture measurement, bottoming out (distance reading) or even auto-calibration for patient immersion or pressure adjustment may also be implemented.

The managing system previously described enables to implement remote maintenance procedures, either for fixing detected issues, either as a predictive maintenance. Such predictive maintenance may be configured based on all data collected on the patient support apparatus during a previous period, or even on all the patient support apparatus of the same model that are used in the same hospital or in other hospitals. This may enable to reduce the time a patient support apparatus is unavailable for care.

According to still a further embodiment, the patient support apparatus may be provided with the same functionalities provided in the previously described web or mobile application. In this embodiment, the application may be implemented directly with the main controller of the patient support apparatus. In such an embodiment, the patient support apparatus becomes an additional access point easing even more the use of the managing system.

As it should now be apparent to the skilled addressee, some aspects of the present technology may provide unmatched precision and reliability, enabling clinical grade precision and reliability (up to 1 centimeter) that are still needed in hospital facilities.

In further embodiments, such clinical grade precision is used for associating and or pairing equipment and/or individuals to predetermined events. Various applications may be implemented. For example, in one embodiment, the system may be used to monitor that a predetermined procedure has been correctly executed. As an example, a cleaning procedure may be monitored, and depending on the duration of the cleaning procedure and real time varying position of the individual performing the task, an algorithm may determine whether the procedure has been conveniently performed or not. Any other predetermined procedure such as medical procedure or maintenance procedure may be monitored. In further embodiments, a specific event or action may be specifically associated with a specific caregiver, even if several caregivers are present in a room in closed vicinity. The medical grade precision will enable to discriminate which one of the caregivers performs the action and/or should be responsible for a predetermined event.

### Location Determination Method

According to a further aspect, there is provided a location determination method for determining a location of a plurality of portable identification devices 200, 300 in a facility, each of the portable identification devices 200, 300 comprising a first portable transceiver and a second portable transceiver, the method comprising: providing a plurality of wireless stationary nodes 100, each of the wireless stationary nodes 100 comprising a processor, a first transceiver and a second transceiver; exchanging first signals between a corresponding wireless stationary node 100 and a corresponding one of the portable identification devices 200, 300 in the vicinity of the wireless stationary node 100: exchanging second signals between the corresponding wireless stationary node 100 and the corresponding one of said portable identification devices 200, 300 in the vicinity of the wireless stationary node 100: and upon reception of the first signals and the second signals by the corresponding one of the wireless stationary nodes 100: determining, by the processor of the corresponding wireless stationary node 100, a precise distance of the corresponding portable identification device 200, 300 from the corresponding wireless stationary node 100 based on the exchanged first signals: determining, by the processor of the corresponding wireless stationary node 100, a precise angle of arrival of the exchanged second signals from the corresponding portable identification device 200, 300 to the corresponding wireless stationary node 100: determining a precise location of the corresponding portable identification device 200, 300 based on the determined precise distance and the determined precise angle of arrival: and providing the determined precise location to a RTLS application 404 for visual representation of the location of each of the portable identification devices 200, 300 in the facility.

The angle of arrival of the received signal may be determined by any suitable method, such as by determining the phase difference of the received signal at two spaced-apart antennas of the receiving device.

In one embodiment, the first signals have UWB signals while the second signals have Bluetooth signals.

In one embodiment, the portable identification devices 200, 300 use only a single transceiver 204 for exchanging signals with the wireless stationary node. In this embodiment, the first and second signals may be of the same type, corresponding to the type of transceiver 204. For example, if the transceiver 204 is a UWB transceiver, the first and second signals are first and second UWB signals. If the transceiver 204 is an ultrasonic transceiver, the first and second signals are first and second ultrasonic signals. In another embodiment, the single transceiver 204 transmits only one signal, for example an UWB signal or an ultrasonic signal, that can be used to determine both the distance to the portable wireless stationary node and the angle of arrival of the signal. The angle of arrival of the received signal may be determined by any suitable method, such as by determining the phase difference of the received signal at two spaced-apart antennas of the receiving device.

In one embodiment, the precise location of each of the portable identification devices 200, 300 is determined using a single one of the wireless stationary nodes 100 closest to the corresponding portable identification device 200, 300 in the facility.

Modifications and improvements to the above-described implementations of the present technology may become apparent to those skilled in the art. The foregoing description is intended to be exemplary rather than limiting. The scope of the present technology is therefore intended to be limited solely by the scope of the appended claims.

1. A method performed by a first device, comprising:
determining, by a first device, that a second device is located in a same room as the first device, based on a first signal communicated between the first device and the second device;
determining, by the first device, a distance to the at least one second device, based on a second signal communicated between the first device and the second device; and
establishing communication with the second device.

2. The method of claim 1, wherein determining, by the first device, a distance to the second device comprises determining a respective distance to each one of a plurality of second devices.

3. The method of claim 1, wherein establishing communication with the second device comprises establishing communication with a nearest one of the plurality of second devices that is located in the same room as the first device.

4. The method of claim 1, wherein the first signal includes a device identifier (ID) corresponding to the second device.

5. The method of claim 4, wherein the device ID is a common device ID to all second devices in the same room.

6. The method of claim 1, wherein the first signal comprises an infrared signal.

7. The method of claim 1, wherein the first signal comprises an ultrasonic signal.

8. The method of any of claims 1 to 7 claim 1, wherein the second signal comprises an ultra-wideband (UWB) signal.

9. The method of claim 1, wherein the second signal comprises an ultrasonic signal.

10. The method of claim 1, wherein the first device is, or is attached to, a piece of medical equipment.

11. The method of claim 10, wherein the first device is, or is attached to, a hospital bed.

12. The method of claim 10, wherein the second device is at a fixed location in the room, and the room is located in a hospital.

13. A first device, comprising:
at least one processor; and
at least one transceiver connected to the processor, the processor being configured to:
determine that a second device is located in a same room as the first device, based on a first signal communicated between the at least one transceiver and the second device;
determine a distance to the at least one second device, based on a second signal communicated between at least one transceiver and the second device; and
establish communication with the second device.

14. The first device of claim 13, wherein determining, by the first device, a distance to the second device comprises determining a respective distance to each one of a plurality of second devices.

15. The first device of claim 14, wherein establishing communication with the second device comprises establishing communication with a nearest one of the plurality of second devices that is located in the same room as the first device.

16. The first device of claim 13, wherein the first signal includes a device identifier (ID) corresponding to the second device.

17. The first device of claim 16, wherein the device ID is a common device ID to all second devices in the same room.

18. The first device of claim 13, wherein the first signal comprises an infrared signal.

19. The first device of claim 13, wherein the first signal comprises an ultrasonic signal.

20. The first device of claim 13, wherein the second signal comprises an ultra-wideband (UWB) signal.

21. The first device of claim 13, wherein the second signal comprises an ultrasonic signal.

22. The first device of claim 13, wherein the first device is, or is attached to, a piece of medical equipment.

23. The first device of claim 22, wherein the first device is, or is attached to, a hospital bed.

24. The first device of claim 22, wherein the second device is at a fixed location in the room, and the room is located in a hospital.

25.-67. (canceled)
